(19) United States
(12) Patent Application Publication
(10) Pub. No.: US 2007/0287800 A1
(43) Pub. Date: Dec. 13, 2007

(54) Lubricious Compounds For Biomedical Applications Using Hydrophilic Polymers

(75) Inventors: Lawrence A. ACQUARULO, Lisbon, CT (US); Charles J. O'NEIL, Chelmsford, MA (US); Amar S. NILAJKAR, N. Las Vegas, NV (US)

Correspondence Address: GROSSMAN, TUCKER, PERREAULT & PFLEGER, PLLC, 55 SOUTH COMMERICAL STREET, MANCHESTER, NH, 03101, US

(73) Assignee: FOSTER CORPORATION, Putnam, CT (US)

(21) Appl. No.: 11/686,779

(22) Filed: Mar. 15, 2007

Related U.S. Application Data
(63) Continuation of application No. PCT/US05/33315, filed on Sep. 15, 2005.
(60) Provisional application No. 60/609,971, filed on Sep. 15, 2004.

(51) Int. Cl. A61K 9/14

(52) U.S. Cl. 525/88; 427/2.3; 525/221

(57) ABSTRACT

The present invention relates to a lubricious polymer compound. The lubricous polymer compound includes a base polymer and a hydrophilic polymer. The lubricous compounds may be injection molded or extruded.

### CROSS REFERENCE TO RELATED APPLICATIONS

This application is a continuation of International Application No. PCT/US2005/033315 filed Sep. 15, 2005 and published Mar. 23, 2006 as International Publication No. WO 2006/032043, designating the United States, and which claims benefit of U.S. Provisional Application No. 60/609,971 filed Sep. 15, 2004, the teachings of which are incorporated herein by reference.

### BACKGROUND

The present invention relates to polymer blends having self lubricating properties. Polymeric materials offer the design engineer unique properties to overcome design challenges in various applications. Advances in various fields like the aerospace industry, automobile industry, telecommunications and biomedical applications like drug delivery, long term implants, etc. would not be possible without the presence of polymers. The use of polymers in biomedical applications has been on a rise since they were first introduced in this field. This has been possible due to the unique combination of properties exhibited by polymers such as flexibility, ease of processing and excellent biocompatibility. Biopolymers are being used in many medical devices involving life saving applications. Artificial implants, drug delivery systems, lubricious coatings for less invasive devices, biological adhesives, anti-thrombogenic coatings and soft tissue replacements are a few of the current commercial applications. Researchers around the world are trying to improve these materials to make them more versatile in their applications with an aim to eliminate the current problems associated with them

Many polymers used in the medical industry lack certain properties. Most polymers exhibit desirable mechanical properties but lack surface properties which are important for processing and performance. The most important properties that are related to the use of polymers in medical devices are wettability and hydrophilicity along with good mechanical properties. Most polymers exhibit poor wettability because of their low surface tension, which is their surface properties. Surface modification techniques can be either physical or chemical in nature. Application of coatings and surface roughening are physical in nature. Plasma treatment and corona discharge are examples of chemical treatment.

An important property for medical devices such as urethral catheters, peripherally centered catheters, urethral stents and catheter sheaths is the ease with which they can be inserted into the body and then removed after the device has performed its required function. Friction between these devices and mucosa can damage the surrounding tissues; hence care should be taken to minimize these effects. It is therefore desirable for the medical devices used inside the body to have as minimum an amount of friction as possible. For design engineers the important properties to be considered in the design of catheters include appropriate mechanical properties to aid insertion and ensure fluid patency, resistance to microbial biofilm formation, resistance to encrustation (in the case of urinary stents/urethral catheters) and lubricity.

Different methods used to achieve surface lubricity are, applying hydrophilic coating to these devices, by surface treatment, using external lubricants or by co-extrusion. All these methods involve a second step operation which does not make it cost effective. Coating operations can create problems during post coating operations like molding the hub on catheter shafts, assembly, welding, etc. Long term stability of these coatings is also being questioned by many researchers, leading to implications that these coatings may not be suitable for implantable devices.

Today hydrophilic polymers are widely being used to modify polymer surfaces in the manufacture of medical devices. These hydrophilic polymers not only enhance lubricity of the polymer surface but also aid in increasing biocompatibility, and control the release of drugs from the medical devices. Literature states that hydrogels or medium cross linked water soluble polymers are known to impart good biocompatibility to different medical devices. This is attributed to the reduced frictional forces between the hydrated material surface and the tissues in the body. Medical devices such as catheters, sheaths, and guide wires require a high degree of surface smoothness to assure introduction into the body without damaging the tissues.

Hydrophilic polymers are presently incorporated into the design of a rich variety of biomedical and pharmaceutical products. Contact lenses, ocular implants, a surfeit of drug delivery systems, lubricious coatings for less invasive devices, biological adhesives, anti-thrombogenic coatings, soft tissue replacements and permanent implants are a few of the current commercial applications that incorporate hydrophilic polymers. Issues related to product feasibility, ease of manufacture, and product-process constraints, as well as environmental and regulatory concerns, all have a direct bearing on the agenda of the engineer when using these materials. A few examples for this family are polyvinyl alcohol, polyethylene glycol, polyvinyl pyrrolidone, cellulosic polymers and polyethylene oxide.

Hydrophilic polymers are unique in their own way, characterized by solubility in and compatibility with water. They are also used in general applications like thickeners in food and paints, coatings for providing static electric dissipation, adhesives in cosmetic formulations and dye receptors. However their unique properties are extensively exploited in the biomedical device industry.

The properties exhibited by these hydrophilic polymers are a direct result of the chemical composition and the molecular structure. The basic bonds include C-C and C-H which are stable in nature. The presence of oxygen (O), nitrogen (N) and hydroxyl group (OH) in the backbone contributes to the water loving nature of these materials. This is represented in FIG. 1. Both O and N are electronegative in nature which results in polymer-solvent interactions of a higher degree. The presence of free electrons allows strong hydrogen bonding with neighboring molecules to occur and may account to some degree for the adhesive properties exhibited by various hydrophilic polymers applied as coatings.

Many of the hydrophilic polymers have polar pendant groups in the vinyl position. These side groups will occur approximately on every other carbon atom in the main chain and contribute greatly to the final properties of the polymer. These side groups are polar and bulky in nature creating a large amount of free volume between the neighboring molecules. This free volume along with polarity allows the water molecules to penetrate in the structure making them hydrophilic. The presence of single covalent bonds in the main chain allows translational and rotational motion when hydrated. The combination of these structural characteristics results in both the dynamic nature and poor mechanical properties. Cross-linking improves the mechanical properties but results in loss of hydrophilicity. A moderate amount of cross-linking gives hydrogels which have balanced mechanical properties along with molecular flexibility and swelling characteristics.

Surface properties can be imparted to medical devices manufactured from conventional polymers by applying hydrogels to them in one way or another. The choice of hydrophilic polymer for a given application will depend on the particular balance of properties required for adequate performance. The most important properties that will be considered are reasonable mechanical properties, degree of swelling, lubriciousness, optical clarity, biocompatibility, pore size and diffusivity.

The ability to characterize the physical properties of the materials used in the fabrication of a given device is of major importance to the process engineer or product design engineer. Knowledge of properties such as tensile strength, modulus, percent crystallinity, glass transition temperature and coefficient of friction serves to improve the quality of the final product or the feasibility of the manufacturing process that will be used to make the product. In the case of hydrophilic polymers, properties like wettability and hydrophilicity along with good mechanical properties play a major role in the final application. These properties will have a direct effect on how the devices perform in a biological environment. Medical devices such as catheters, sheaths, and guide wires require a high degree of surface smoothness to assure introduction into the body without damaging the tissues.

The definition of wettability as provided by Zisman is the ability of a liquid to adhere to a solid and spread over its surface to varying degrees. Wettability is often referred to as hydrophilicity but is considered to be a surface property as opposed to true hydrophilicity which is considered as a bulk property. The degree of wettability depends on the intended application. For example, in applications where moisture resistance is required, minimum wettability is desired so that water will not adhere to the substrate. In contrast, for adhesive applications, maximum wettability is required.

For medical devices which come into contact with blood and tissue, it is desirable that the materials used have a higher degree of wettability or hydrophilicity. The reason for this is that the biological environment is hydrophilic in nature and biocompatibility appears to correlate directly with the degree of hydrophilicity of the surface. Most polymers used in biomedical applications are hydrophobic in nature, hence the idea of using hydrophilic polymers along with other polymers in making biomedical devices. These hydrophilic polymers have very limited mechanical properties, due to which they are restricted in applications. They are currently being used extensively as coatings for many medical devices for imparting wettability to the surface. Coating application is generally solvent based, which may lead to non-uniform spreading if applied incorrectly. In this research project use of these hydrophilic polymers was considered in a different way by the process of melt blending.

When one solid body is slid over another there is resistance to motion which is called friction. It is usually considered that friction is a nuisance and from earliest times man has made attempts to eliminate it or to diminish it to the smallest value as possible. For the use of plastics in various applications, it is always desired to reduce friction and eliminate wear of the components involved. In medical applications the friction associated with the medical device results in the damage of tissues surrounding it in the body. This has generated a lot of interest among materials scientists to eliminate this problem or reduce it. The basic material property used for the development of medical devices is the coefficient of friction. For a pair of surfaces, the ratio of friction to load is constant, and this constant is called the “coefficient of friction”. The coefficient of friction varies widely with different polymers. It is always desired to have low coefficient of friction for polymers used in medical applications.

The equation shows the relationship between friction and load (N) where μ represents the coefficient of friction. We have two types of friction, namely static and dynamic (kinetic), represented by μₛ and μₖ. FIG. 2 gives an explanation for the different types of friction.

In the development of medical devices like catheters, lubricity is often determined using conventional frictional tests based on ASTM standards. However, this test does not simulate the wet conditions in the body. Many researchers have developed their own methods to determine lubricity in the biological environment seen in the body. Marmieri et al developed a good method which is being used by others as a reference. A sample catheter material is inserted in a model biological medium, agar, which simulates the humid, moist environment in the body, and a weight is employed to pull it. The time taken to pull the sample out of the medium is correlated to the slipperiness of the material. Lubricious materials tend to be removed quickly from the medium whereas a longer period of time is required to remove more frictional materials. Jones et al describes a method that employs a texture analyzer to characterize the force required to insert catheters and remove them from model substrates. In the present study, friction in the dry state was used to characterize different materials.

Since the invention of high impact polystyrene there has been a great deal of activity on innovative polymer blends to develop synergistic properties, and on innovative blending processes to maximize their unique characteristics. Today polymer blends are of considerable interest and present great challenges to the research scientist. The applications range for these materials is vast and new technologies with various polymers are emerging. Applications requiring a balance of properties, including costs, beyond those contributed by the individual polymers, have catalyzed the exploration, development and commercialization of several novel polymer blends. During the past 50 years the growth of polymer blend technology has been explosive. New inventions and innovations in blends have developed into a science and resulted in the growth of the plastics industry resulting in many new applications.

Polymer blends do not usually form homogeneous mixtures but show micro or macro-phase separation. This immiscibility has some inherent advantages as well as disadvantages when compared to the individual components. Materials with different properties and structures can be obtained by varying the composition as well as the processing conditions. The final properties may be far superior to the individual components. In general terms polymer blends can be defined as “a combination of two or more polymers resulting from common processing steps such as mechanical blending, solution casting or in some cases chemical synthesis”. Graft copolymers and block-copolymers as well as cross linked polymers, do not come under this definition but may be similar in properties to the polymer blends.

The preparation technique for blends is most important from the economical point of view as well as the final properties. The challenges in blending the high molecular weight polymers, most of them being immiscible, have contributed to several innovations resulting in novel technologies and patents. The following techniques are generally used for manufacturing polymer blends.

Different polymers are dissolved in a common solvent and then cast. The resulting product is a film. Limitations of this method are that not all polymers are readily soluble in common and safe solvents. Thick shapes cannot be cast easily, and the residual solvent can affect the blend properties. The nature of the final product can depend strongly on the type of solvent used and the casting conditions. Example: PS/PMMA from toluene.

Here a solution of the two polymers is quenched down to a very low temperature and the solvent is frozen. Solvent is later removed by sublimation. In most cases the resulting blend will be independent of the solvent if the solution is single phase and the freezing occurs rapidly. The disadvantages with this method are that solvents used must be symmetric like benzene, naphthalene, etc. Large quantities cannot be processed and the powdery form of the blend after solvent removal has to be reshaped. Example: PS/PMMA in naphthalene.

This includes exchange reactions between components, polymerization of a monomer in the presence of second polymer, IPN formation and co-crosslinking. In most cases reaction can be controlled and good homogeneous systems can be prepared. Example: LDPE/PS Melt state mixing is the most widely used technique for making polymer blends. This technique was used in this research work and is explained in detail in the following paragraphs.

By using the right equipment, different polymers can be blended in their molten state, giving good dispersion and equilibrium of the components. It is a cost effective technique, with no solvents or any other foreign components involved. It is a fast process and the temperature and environment can be controlled easily to make good blends. Various instruments used to do this are the two roll mill, banbury mixers, single and twin screw extruders. The most widely used technique for melt blending is by single and twin screw extrusion. Different types of screws can be used to get proper mixing of two polymers leading to the formation of compatible blends.

The degree of compatibility between two or more polymers in blends may vary based on various factors like processing conditions, polarity etc. and in most cases polymer pairs are not miscible on the molecular level. When a very close match in cohesive energy density is seen, or when the polymers involved can co-crystallize, miscibility in such systems can be observed. Along with the possibility of forming separate phases with various sizes, shape and geometrical arrangements, more complex structures are possible. The most important area of study for these blends is the dependence of mechanical properties on the composition. These complex systems exhibit behavior quite different from the individual components and do not simply follow the sum of the properties of the components.

The compatibility or incompatibility of polymer blends is ultimately related to the practical applications. Some mechanical properties of compatible systems may deviate only to a small extent from those anticipated on the basis of a linearly additive scheme. In contrast, the properties of immiscible blends may be governed by the more dominating polymer involved, or the properties may actually lie outside the two or more polymers present in the system. The traditional experimental criteria for compatibility have been extensively reviewed elsewhere as explained by Newman. Among all the techniques, glass transition behavior has prevailed as a diagnostic test of miscibility. This has been possible due to technological improvement of Tg measuring instruments which are more sensitive to changes.

Polymers can have both amorphous and crystalline regions. Based on the temperature, the amorphous regions can be either in the glassy or rubbery state. “The temperature at which the transition in the amorphous regions between the glassy and rubbery state occurs is called the glass transition temperature”. A large variety of measurement techniques are available to measure the glass transition temperature. These techniques include calorimetric determination of heat capacities as a function of temperature, dynamic mechanical measurements of complex modulus as a function of temperature, dielectric relaxation spectroscopy, dilatometry, etc. The first two techniques are the most prominently used for measuring Tg. The literature mentions that Tg measurement is subject to certain limitations which are outlined below.

If the component Tg's cannot be differentiated the test fails to discriminate between miscible and immiscible blends. This is dependent on the individual components, and if the difference in Tg's lies within 20° C. or less of each other, it is difficult to determine the miscibility.

If the detectibility of Tg of either component is reduced due to its lower concentration in the mixture, the use of the single Tg test to determine compatibility will be compromised. If either of the components is crystallizable, it becomes difficult to distinguish between the glass transition and melting transition that take place in the blend. It can be said that Tg measurement is sensitive to the concentration of the polymers and their physical state.

Differential scanning calorimetry or DSC is a widely used thermal analysis technique for plastics. It is helpful for quality control and basic material characterization. This technique measures the quantity of energy absorbed or given off by a sample in calories as its temperature is changed. The sample and an inert reference are heated at a constant programmed rate and the difference in energy required to heat the two samples is measured. A schematic representation of the method is shown in FIG. 3 below. In the transition regions more or less energy is used by the samples depending on whether the process is endothermic or exothermic in nature. For example, when a sample melts, it uses more energy than the reference and the process is endothermic. A typical DSC curve is shown in FIG. 4 which indicates the glass transition temperature (Tg), melting point (Tm) and crystallization temperature (Tc). In polymer characterization, DSC helps in studying the melting behavior, degree of cure, melting point determination, oxidative stability, Tg determination, degradation behavior and miscibility of polymer blends.

The advantage of DSC is that it has small sample requirements, relatively rapid measurement capability and high sensitivity. A discontinuity seen in the specific heat (Cp) vs. temperature curve represents the glass transition temperature.

Mechanical properties of plastics are very important because most of the end-use applications involve some degree of mechanical loading. The selection of materials for any kind of application begins with the knowledge of mechanical properties such as tensile strength, yield strength, flexural modulus, elongation and impact properties. For most commercial polymers these values are provided by the manufacturer is literature. These properties are generated by carrying out tests at laboratory conditions. In most applications the polymers involved are affected by many conditions like the environment and temperature, etc; hence the final selection of the material must consider all these parameters. Mechanical properties obtained by testing under standard laboratory conditions help to eliminate many materials from a large group.

In this work, mechanical properties were used to identify the most suitable materials for the intended application. The effect of blend ratio on the final properties was studied. Medical devices require good tensile strength and yield strength along with stiffness. These properties are very important for applications like catheters and tubing, where the device undergoes deformation during insertion.

Rheological properties are very important for processing of polymers using injection molding, extrusion and other polymer melt processing operations. Apparent viscosity is measured in a capillary rheometer over an entire range of shear rates encountered in the above mentioned melt processing operations. The viscosity of the melt can be used to determine the melt behavior of different polymers in the mold for injection molding operations or through the die for extrusion. Viscosity data is very important in making devices such as catheters and tubes using extrusion. If the viscosity is very low, profiles cannot be made due to insufficient melt strength of the polymer. This property is also useful for research and development and quality control purposes.

Thermoplastic elastomers (TPEs) are a class of materials that exhibit properties of elasticity and rubber like behavior in addition to thermoplastic behavior. These materials can be processed on conventional thermoplastics processing equipment. They are broadly classified into five conventional classes which include styrenics, polyolefins, copolyesters, polyurethanes and the polyamide based TPEs. An in-depth explanation of each class is avoided in this discussion and a description of only those materials pertinent to the topic is included. Although there is a certain amount of overlap in applications and properties of various TPEs each class has a certain application market because of its unique properties and advantages.

These materials have unique properties because of their basic structure. TPEs are made up of soft and hard segments. The soft segments contribute to the flexibility and extensibility of the elastomer. The glassy or semi-crystalline hard segment serve as virtual crosslinks. The hard phase contributes to the physical properties as well as oil and solvent resistance of the TPE. These crosslinks are physical in nature in contrast to the chemical bonds in vulcanized rubber, and are therefore thermally reversible. FIG. 5 shows a representation of the hard and soft segments seen in thermoplastic elastomers. Phase mixing of the hard and soft segments occurs above the melting point of the hard segments and they can be processed in conventional thermoplastic machines. Upon cooling, the hard and soft segments become immiscible again and phase separate, leading to the reformation of physical crosslinks.

In the evaluation and development of medical devices, the choice of the material is only one of the factors that must be considered. The potential to integrate a device into the biological environment is of increasing importance. In this regard the use of thermoplastic elastomers in medical applications has been growing recently. This has been possible due to the unique combination of mechanical properties and biocompatibility provided by these materials. Different applications seen for these materials are outlined below.

Catheters are widely used in medical applications for therapeutic and diagnostic purposes. They are used for the delivery and removal of fluids from the body. They are also used for more complex applications like angioplasty and as the insulating sheath of pacemaker leads. The main factors to be considered for evaluating the performance of catheters are the roughness of the surface, susceptibility to colonization by bacteria, stiffness and flexibility to allow easy insertion. FIG. 6 shows a general picture for a catheter. FIG. 7 shows pictures of catheters used in angioplasty. The tip of the catheter has a balloon which expands when air is introduced into the device and this helps in opening of the veins and arteries.

The development of artificial organs for biomedical applications is necessary to perform the functions of organs that are diseased and no longer function adequately. Artificial hearts, pancreas, kidneys, blood tubing and oxygenators are a few of the artificial organs made from thermoplastic elastomers. The main properties that are required of materials for these applications are good biocompatibility on the inside surface, good tissue compatibility on the outside surface and infection resistance. Good flexural properties and mechanical strength are critical to the performance.

These materials have been used successfully in breast implant devices and implants in dentistry, adhesives, urology, cardiology and wound dressings. For breast implants the materials must have mechanical properties which mimic the human breast; they need to be flexible and deformable while maintaining the appropriate shape. The use of thermoplastic elastomers in wound dressing applications is possible due to their good barrier properties and oxygen permeability to facilitate cell growth.

Other applications include artificial ducts, contraceptives, controlled drug delivery, ligament replacements, nerve guides and invertebral discs.

Thermoplastic polyurethane elastomers (TPUs) were the first homogeneous thermoplastically processable elastomers and today they play an important role within the rapidly growing family of thermoplastic elastomers.

TPUs are made from long chain polyols with an average molecular weight of 600 to 4000 along with polyisocyanates and chain extenders with a molecular weight of 61 to 400. Due to the wide range of hard to soft segment variation, TPUs can be formulated to form soft flexible elastomeric materials to more brittle high modulus plastics. Soft segments form the elastomeric matrix which gives the elastic properties to TPU. This phase controls the low temperature properties, and resistance to solvents, of TPUs.

The most important flexible segments are formed using either hydroxyl terminated polyesters or hydroxyl terminated polyethers. Hard segments act as physical crosslinks and reinforcing fillers, controlling the mechanical properties. Polyisocyanates are the most commonly used hard segments. The choice of chain extenders and diisocyanates used determines the characteristics of the hard segments and the overall properties of TPUs. The most important chain extenders used for TPUs are linear diols such as ethylene glycol. A typical structure of TPU is shown in FIG. 8 below. The properties of a TPU include resistance to abrasion, puncture and tear propagation, excellent bondability and weldability, excellent mechanical properties, good resistance to hydrolysis and microbes, good impact resistance at low temperatures, good chemical resistance and weathering properties, good biocompatibility.

Currently TPUs are widely used in many medical devices. These materials show good compatibility to human skin. This biocompatibility allows TPUs to be used in making catheters and tubing for medical diagnosis and many other medical devices.

Polyamide based TPEs were first developed in the 1980's by Dow Chemicals and by Atochem. The four major types of these elastomers are the polyesteramides (PEAs), polyetheresteramides (PEEAs), polycarbonate-esteramides (PCEAs) and polyether-block-amides (PE-b-As).

The hard segments are based on aliphatic amides and semi-aromatic amides while the soft segments are based on aliphatic polyesters, aliphatic polyethers or aliphatic polycarbonates. A typical structure is shown in FIG. 9 below. Properties of a polyamide based TPE include god tensile properties, excellent chemical and solvent resistance, good tear strength and abrasion resistance, good adhesive properties and weatherability, low compression set, and good thermal ageing and high service temperatures.

Polyamide based TPEs are the newest addition to the class of TPEs and their full range of applications is yet to be discovered. Because of their higher service temperatures and good thermal aging these TPEs are expected to fill the gap between the thermoplastic polyurethanes and the silicone based polymers. The balance of these properties allows them to be used for under the hood applications in the automobile industry. Another application is for high temperature insulation in the wire and cable industry. Many specialized applications such as antistatic packaging or humidity-sensors can be achieved by using tailor made polyamide elastomers. The latest applications for this class are in the medical device industry. These materials have excellent biocompatibility and good mechanical properties which make them highly unique in the medical field.

Nylon 12 is manufactured using laurolactam as the monomer which is derived from butadiene. The structure of nylon 12 is given below in FIG. 10. The amide groups help in the formation of hydrogen bonding and are responsible for all the properties. These bonds are responsible for crystallinity, increase the strength, melting point and chemical resistance. The concentration of amide groups is the lowest in nylon 12 and is responsible for some of its unique properties which include lowest moisture absorption, i.e. properties are not affected by moisture, excellent solvent resistance and resistance to stress cracking, excellent impact strength, dimensional stability, low coefficient of friction.

Polyolefins are very widely used both as elastomers and rigid thermoplastics. Due to their attributes of chemical inertness, low density, and low cost they offer major advantages over many other polymers. There are two distinct types of polyolefin blends that are TPEs. They are the co-continuous phase blends and dynamically vulcanized blends.

In the co-continuous phase blends both the elastomeric phase and the crystalline polyolefin phase are continuous. Both these phases flow during processing and the elastic properties depend mostly on the crystalline polyolefin phase. Dynamically vulcanized blends have the semicrystalline polyolefin phase as continuous and the crosslinked elastomeric phase is discontinuous. Elastic properties are dependent on both the continuous polyolefin phase and the chemically crosslinked elastomer phase. Properties include good mechanical properties, low density and cost, good chemical inertness, good biocompatibility, ease of processing, good weatherability.

TPOs are widely used in automotive applications, wire and cable industry and mechanical goods. Their unique properties combined with low cost make them suitable for interior and exterior automotive parts like bumper covers, air dams, conduit, etc. Excellent electrical properties, water resistance and ozone resistance make them suitable for wire and cable applications. They are used for making flexible cords, booster cables, appliance wire and low voltage jacketing. The latest applications include the medical device industry, due to their good mechanical properties, surface smoothness, biocompatibility and low cost.

A brief description of the different hydrophilic polymers being used in this study is outlined below. Ethyl cellulose belongs to the class of thermoplastic cellulose ethers. It was first introduced by Dow in the mid 1930's and is widely used for many applications today. A reaction between ethyl chloride and alkali cellulose results in the formation of ethyl cellulose. These polymers possess performance and compatibility that is unique and economically advantageous. Ethyl cellulose has a polymeric backbone chain consisting of cellulose. The structure of cellulose is shown in FIG. 11 below. Each repeating unit has three reactive hydroxyl sites which can be replaced by ethoxyl group upon etherification reaction leading to the formation of ethyl cellulose. The structure of ethyl cellulose is shown in FIG. 12. Physical properties vary depending upon the degree of etherification (replacement of the hydroxyl groups by ethoxyl groups). The presence of hydroxyl groups on the backbone makes it hydrophilic in nature. Properties include good dimensional stability, low temperature properties, heat stability and low ash content, compatibility with other resins and plasticizers, and good biocompatibility.

Applications include adhesives and coatings for medical applications, electrical insulation and fabric coating, printing inks and varnishes, controlled drug release and granulation aids and tablet binders Polyethylene glycol is a linear homopolymer derived from the monomer ethylene oxide. They are waxy materials available in different molecular weight grades. They are commercially known as Carbowax, Polyox, etc. They are soluble in water and many organic solvents. They find application in pharmaceutical applications and cosmetics. An ideal structure of polyethylene glycol is shown in FIG. 13.

Polyvinyl pyrrolidone is a synthetic water soluble polymer made up of N-vinyl pyrrolidone as the repeating unit. Pyrogen and pyrogen free grades are available. This polymer finds excellent use in medical applications, oral care and as an additive in many dye compositions. The general structure is shown in FIG. 14.

Polyethyl oxazoline has excellent water solubility, and thermal stability makes it a preferred substitute for polyvinyl alcohol and polyvinyl pyrrolidone in high temperature applications. A general structure is shown in FIG. 15. Currently, it is used in a variety of hot-melt and pressure-sensitive adhesive products. It also finds use in the ceramics industry as a greenware binder because of the clean burn-out and non-ionic nature of this polymer. Other applications include coatings, textile and fiberglass sizing, lubricants, plasticizers, compatibilizers and films.

### SUMMARY

An exemplary embodiment of the present invention relates to a lubricious polymer compound comprising hydrophilic polymers that are dispersed in a melt processable thermoplastic matrix.

Another exemplary embodiment of the present invention relates to a lubricious polymer compounds comprising a hydrophilic polymer dispersed in a melt processable thermoplastic matrix having a static and dynamic coefficient of friction values of about 0.02 or greater.

Another exemplary embodiment of the present invention relates to a catheter comprising a tube including a layer of a lubricous polymer compound comprising a hydrophilic polymer dispersed in a melt processable thermoplastic matrix.

Another exemplary embodiment of the present invention relates to a wire comprising a surface layer of a lubricous polymer compound comprising a hydrophilic polymer dispersed in a melt processable thermoplastic matrix.

Another exemplary embodiment of the present invention relates to a lubricious polymer compound comprising hydrophilic polymers that are dispersed in a melt processable thermoplastic matrix including a hydrophilic polymer dispersed on the surface of said compound by solution casting.

Another exemplary embodiment of the present invention relates to a lubricious polymer compound comprising a base polymer and a hydrophilic polymeric as a layer on extruded tubing.

Another exemplary embodiment of the present invention relates to a method of applying a lubricious polymer compound comprising a hydrophilic polymer dispersed in a melt processable thermoplastic matrix comprising supplying said lubricious polymer compound; and extruding said lubricious polymer compound on to a surface of a thermoplastic resin substrate.

### BRIEF DESCRIPTION OF THE DRAWINGS

FIG. 1 depicts examples of chemical and molecular structures of hydrophilic polymers.

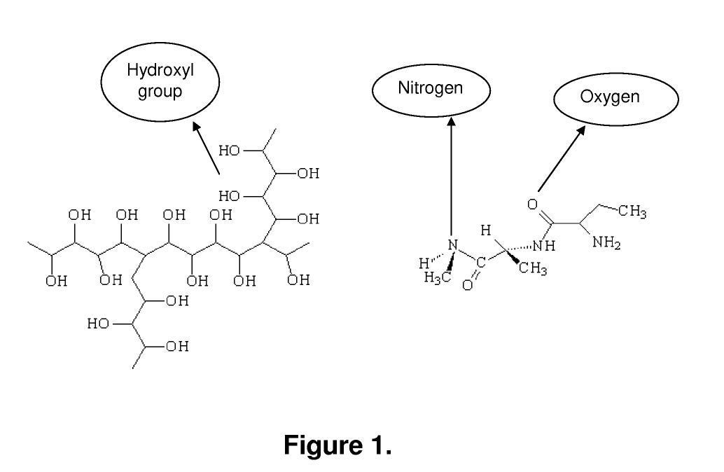

FIG. 2 depicts an exemplary schematic representation of friction.

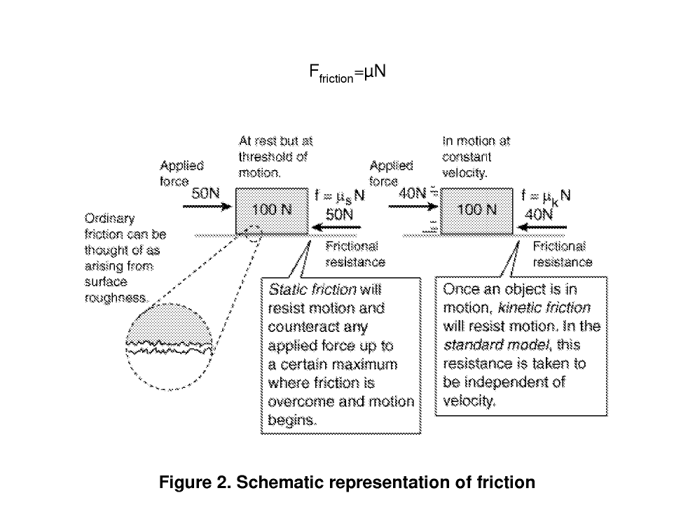

FIG. 3 depicts a schematic representation of an exemplary DSC (Differential Scanning Calorimetry) test method.

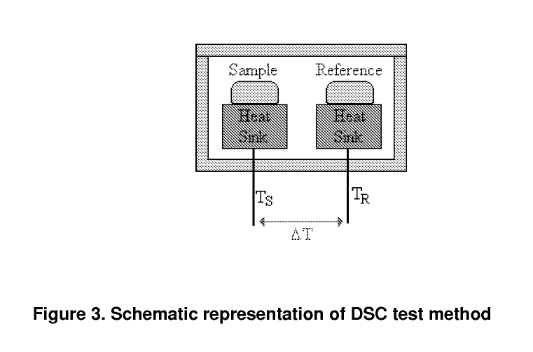

FIG. 4 depicts a typical DSC curve for polymers.

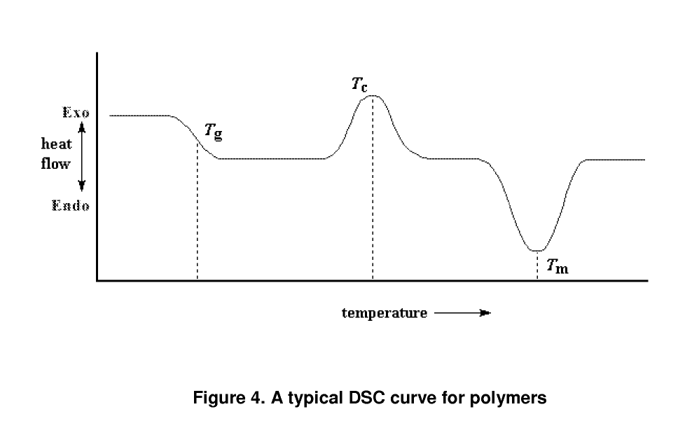

FIG. 5 depicts a schematic representation of an example of a thermoplastic elastomer.

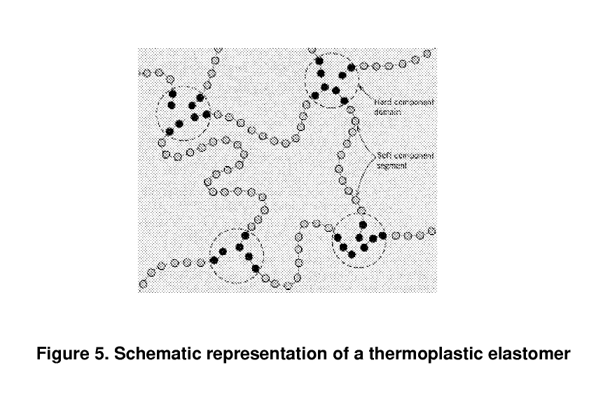

FIG. 6 depicts a typical catheter.

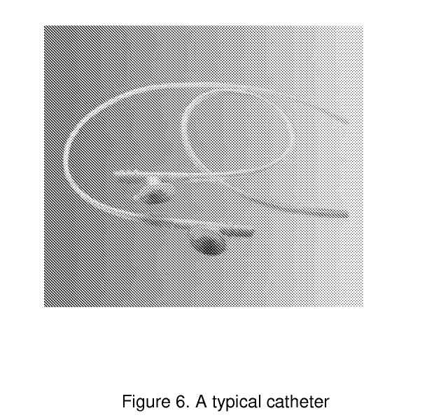

FIGS. 7A and 7B depict examples of catheter systems with balloons at their tips for angioplasty.

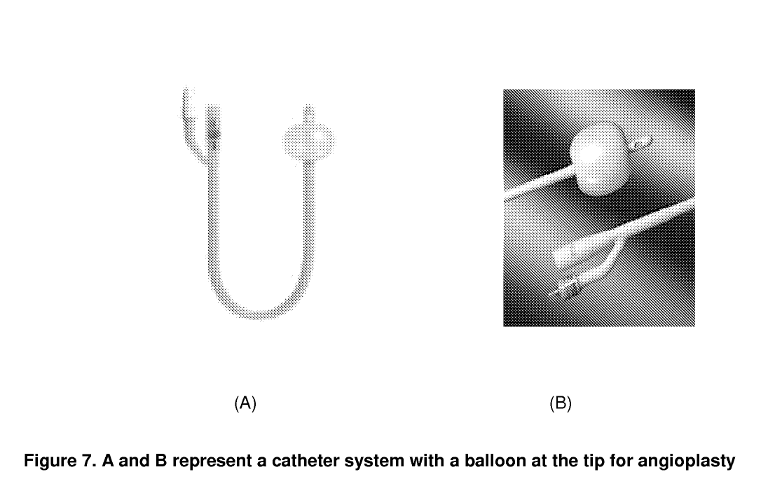

FIG. 8 depicts a typical structure of a TPU (Thermoplastic Polyurethane Elastomer).

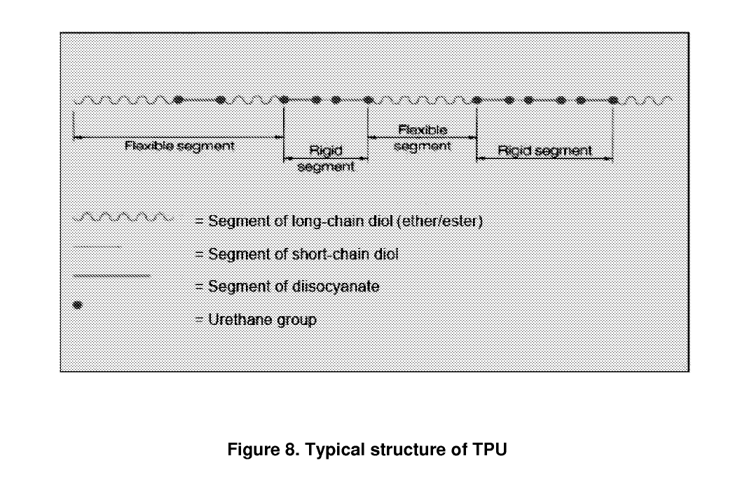

FIG. 9 depicts a typical structure of a Polyamide based TPE (Thermoplastic Elastomer).

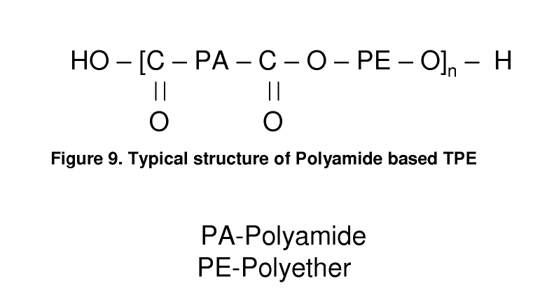

FIG. 10 depicts a chemical structure of Nylon 12.

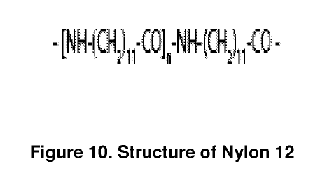

FIG. 11 depicts an example of a molecular structure of cellulose.

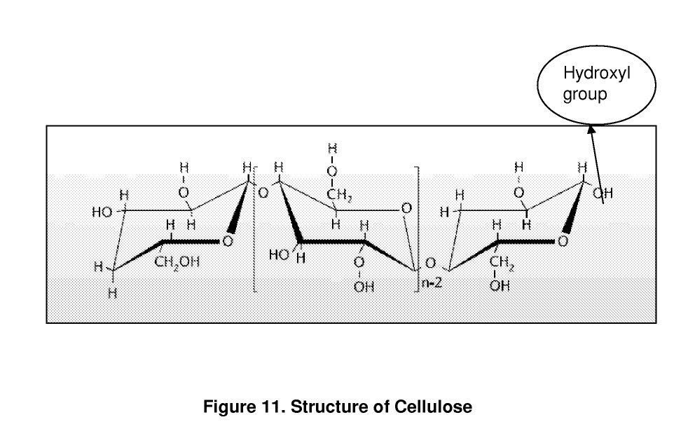

FIG. 12 depicts an example of a molecular structure of Ethyl cellulose.

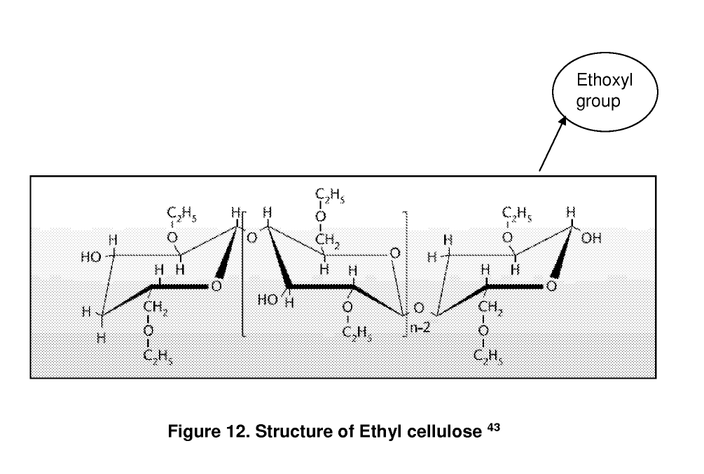

FIG. 13 depicts an example of an ideal structure of Polyethylene oxide.

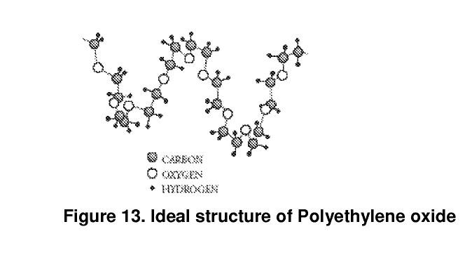

FIG. 14 depicts a general molecular structure of Polyvinyl pyrrolidone.

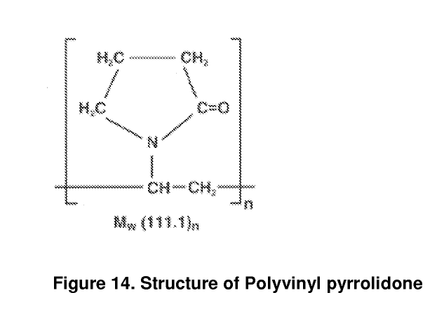

FIG. 15 depicts a general molecular structure of Polyethyl oxazoline.

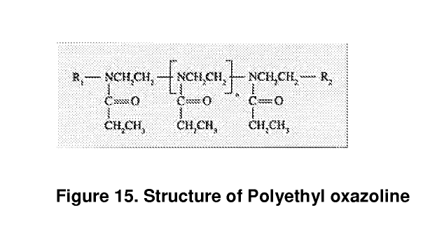

FIG. 16 depicts an exemplary block diagram consistent with the present disclosure.

FIG. 17 depicts an exemplary bar graph illustrating the effect of different hydrophilic polymers on break strength for Pellethane.

FIG. 18 depicts an exemplary bar graph illustrating the effect of different hydrophilic polymers on percent strain at break for Pellethane.

FIG. 19 depicts an exemplary bar graph illustrating the effect of different hydrophilic polymers on tensile modulus for Pellethane.

FIG. 20 depicts an exemplary bar graph illustrating the effect of different hydrophilic polymers on flex modulus for Pellethane.

FIG. 21 depicts an exemplary bar graph illustrating the effect of different hydrophilic polymers on the static coefficient of friction for Pellethane.

FIG. 22 depicts an exemplary bar graph illustrating the effect of different hydrophilic polymers on the dynamic coefficient of friction for Pellethane.

FIG. 23 depicts an exemplary bar graph illustrating the effect of different hydrophilic polymers on viscosity for Pellethane.

FIG. 24 depicts exemplary plots illustrating the effect of different hydrophilic polymers on viscosity for Pellethane.

FIG. 25 depicts a comparison of DSC plots for Pellethane and its exemplary blends.

FIG. 26 depicts an exemplary bar graph illustrating the effect of different hydrophilic polymers on yield strength for Pebax.

FIG. 27 depicts an exemplary bar graph illustrating the effect of different hydrophilic polymers on percent strain at yield for Pebax.

FIG. 28 depicts an exemplary bar graph illustrating the effect of different hydrophilic polymers on break strength for Pebax.

FIG. 29 depicts an exemplary bar graph illustrating the effect of different hydrophilic polymers on percent strain at break for Pebax.

FIG. 30 depicts an exemplary bar graph illustrating the effect of different hydrophilic polymers on tensile modulus for Pebax.

FIG. 31 depicts an exemplary bar graph illustrating the effect of different hydrophilic polymers on flex modulus for Pebax.

FIG. 32 depicts an exemplary bar graph illustrating the effect of different hydrophilic polymers on static coefficient of friction for Pebax.

FIG. 33 depicts an exemplary bar graph illustrating the effect of different hydrophilic polymers on dynamic coefficient of friction for Pebax.

FIG. 34 depicts an exemplary bar graph illustrating the effect of different hydrophilic polymers on viscosity for Pebax.

FIG. 35 depicts exemplary plots illustrating the effect of different hydrophilic polymers on viscosity for Pebax.

FIG. 36 depicts a comparison of DSC plots for Pebax and its exemplary blends.

FIG. 37 depicts an exemplary bar graph illustrating the effect of different hydrophilic polymers on yield strength for Vestamid.

FIG. 38 depicts an exemplary bar graph illustrating the effect of different hydrophilic polymers on percent strain at yield for Vestamid.

FIG. 39 depicts an exemplary bar graph illustrating the effect of different hydrophilic polymers on break strength for Vestamid.

FIG. 40 depicts an exemplary bar graph illustrating the effect of different hydrophilic polymers on percent strain at break for Vestamid.

FIG. 41 depicts an exemplary bar graph illustrating the effect of different hydrophilic polymers on tensile modulus for Vestamid.

FIG. 42 depicts an exemplary bar graph illustrating the effect of different hydrophilic polymers on flex modulus for Vestamid.

FIG. 43 depicts an exemplary bar graph illustrating the effect of different hydrophilic polymers on static coefficient of friction for Vestamid.

FIG. 44 depicts an exemplary bar graph illustrating the effect of different hydrophilic polymers on dynamic coefficient of friction for Vestamid.

FIG. 45 depicts an exemplary bar graph illustrating the effect of different hydrophilic polymers on viscosity for Vestamid.

FIG. 46 depicts exemplary plots illustrating the effect of different hydrophilic polymers on viscosity for Vestamid.

FIG. 47 depicts a comparison of DSC plots for Vestamid and its exemplary blends.

FIG. 48 depicts an exemplary bar graph illustrating a comparison of impact strength for Vestamid and its exemplary blends.

FIG. 49 depicts an exemplary bar graph illustrating the effect of different hydrophilic polymers on yield strength for Marlex.

FIG. 50 depicts an exemplary bar graph illustrating the effect of different hydrophilic polymers on percent strain at yield for Marlex.

FIG. 51 depicts an exemplary bar graph illustrating the effect of different hydrophilic polymers on break strength for Marlex.

FIG. 52 depicts an exemplary bar graph illustrating the effect of different hydrophilic polymers on percent strain at break for Marlex.

FIG. 53 depicts an exemplary bar graph illustrating the effect of different hydrophilic polymers on tensile modulus for Marlex.

FIG. 54 depicts an exemplary bar graph illustrating the effect of different hydrophilic polymers on flex modulus for Marlex.

FIG. 55 depicts an exemplary bar graph illustrating the effect of different hydrophilic polymers on static coefficient of friction for Marlex.

FIG. 56 depicts an exemplary bar graph illustrating the effect of different hydrophilic polymers on dynamic coefficient of friction for Marlex.

FIG. 57 depicts an exemplary bar graph illustrating the effect of different hydrophilic polymers on viscosity for Marlex.

FIG. 58 depicts exemplary plots illustrating the effect of different hydrophilic polymers on viscosity for Marlex.

FIG. 59 depicts a comparison of DSC plots for Marlex and its exemplary blends.

FIG. 60 depicts an exemplary bar graph illustrating the effect of different hydrophilic polymers on impact strength for Marlex.

### DETAILED DESCRIPTION

The approach used here is to melt blend various base polymers used in the medical industry with hydrophilic polymers known to impart lubricity when applied on the surface as hydrophilic coatings. Use of these hydrophilic polymers alone is limited by the fact that they become mechanically weak in the presence of water; hence the approach of melt blending has been used to give a good combination of mechanical properties and surface properties.

In one embodiment, the lubricious polymer compounds may be used as a single layer or as an inner layer or outer layer in a coextruded tube. The lubricious polymer compounds may contain hydrophilic polymers that are dispersed in a melt processable thermoplastic matrix. The lubricious polymer compounds as described herein may be processed using conventional thermoplastic processing equipment such as extrusion and molding.

The lubricious polymer compounds may contain hydrophilic polymers, including, for example, polyethylene glycol, polyvinyl pyrrolidone, polyethyl oxazoline or ethyl cellulose. The lubricious polymer compounds containing hydrophilic polymers may be blended with a thermoplastic matrix such as thermoplastic polyurethane elastomers, polyether block copolyamide polymers, polyamide 12 and polyethylene.

The lubricious polymer compounds has a static and dynamic coefficient of friction values as low as 0.02. For example, the lubricious polymer compounds when applied to the inside of a tube may exhibit the characteristics common of a PTFE liner in such catheters as a guide catheter, or in a push/pull cable or in a steering cable.

The lubricious polymer compounds may be extruded over a tube core, or solid or braided wire or guide wire core, and may in addition to or thereby eliminate the need for a secondary operation where a solution coating is applied. Furthermore, the lubricious polymer compounds that could be used as a tie layer to improve the adhesion of a surface coating on extrusions.

The lubricious polymer compounds used in a one-step coextrusion process in which the lubricated inner or outer layer is achieved in a single step and may thereby eliminate the need of a post-processing operation such as solution coating. The lubricious polymer compounds may also be used in a mono-layer extruded product in the above mentioned applications including for example, tubing or coatings used in medical applications such as catheters or tubing coatings.

The lubricious polymer compounds may furthermore have sufficient integrity as not to rub off during handling, assembly and in use in the above mentioned applications. The lubricious polymer compounds may also maintain the characteristics of the thermoplastic matrix allowing for sufficient bonding in post-extrusion operations such as welding, adhesive bonding, over-molding, etc. Furthermore, the lubricious polymer compounds when used in combination with a solution hydrophilic polymer coating may improve the reliability by maintaining sufficient lubricity providing redundant lubricious characteristics in the event the said lubricious coating wears off.

Different base polymers may be used including polyurethane, nylons and polyethylene based thermoplastic elastomers which are widely used in the medical device industry. Hydrophilic polymers being used include, for example, polyethylene oxide, polyethylene glycol, ethyl cellulose and polyvinyl pyrrolidone. These polymers are known to have different degree of solubility in the presence of water or fluids containing water. The main objective here is to achieve a good combination of mechanical properties of the base polymers and beneficial biocompatible properties of the hydrophilic polymers by melt blending the two. These polymers may be melt-blended at concentrations of 25% and 50% using a twin screw extruder. However, various other blending techniques may be employed such as single screw extrusion, injection molding, mixing in a mixer, etc.

A number of non-limiting examples of the present invention are described herein. These examples are non-limiting descriptions of the present invention and a person of ordinary skill in the art would understand that other hydrophilic material and base polymer blends may be within the scope of the present invention.

Table 1 gives a list of materials. The basic properties as reported for the base polymers and hydrophilic polymers are shown in Table 2 and Table 3 respectively. Three different grades of Polyethyl oxazoline were used in the ratio of 1:2:2 as shown in the Table 1.

TABLE 1
List of Materials used

| Type | Family | Manufacturer | Trade name | Grade |
|---|---|---|---|---|
| Base | Polyurethane TPE | Dow Chemicals | Pellethane ® | 2363-80 A |
| polymers | Polyamide TPE | Atofina | Pebax ® | 2533 |
| | Nylon 12 | Degussa | Vestamid ® | L2140 |
| | Polyolefin | Chevron Phillips | Marlex ® | 5202 BN |
| Hydrophilic | Ethyl Cellulose | Dow Chemicals | Ethocel ® | Standard 100 |
| polymers | Polyvinyl Pyrrolidone | ISP technologies | Plasdone ® | C-30 |
| | | Inc | | |
| | Polyethylene Glycol | Dow Chemicals | Polyox ® | WSR-301 |
| | Polyethyl oxazoline | Polymer Chemistry | Aquazol ® | 50/200/500 |
| | | Innovations | | (10%/20%20%) |

TABLE 2
Basic Properties of the Base Materials used*

| Property type | Pellethane | Pebax | Vestamid | Marlex |
|---|---|---|---|---|
| Specific Gravity | 1.13 | 1.01 | 1.01 | 0.95 |
| Hardness | 81 Shore A | 25 Shore D | NA | NA |
| Melting point (° C.) | 193-210 | 148 | 178 | 130 |
| Glass transition | −42 | −65 | 30-40 | −117 |
| temperature (° C.) | | | | |
| Flex Modulus | NA | 15 | NA | 1309 |
| (MPa) | | | | |
| Tensile Strength | 12.1 at | 34 | 48 | 27 |
| (MPa) | 300% | (Ultimate) | (at yield) | (at yield) |
| % Elongation | 550 | 640 | >50 | 600 |
| Melt index | 23 | 14 | 36 | 0.35 |
| (g/10 min) | | | | |
| Notched charpy | NB | NB | 1.1 J/cm² | NA |
| impact strength | | | | |

TABLE 3
Basic Properties of the Hydrophilic Materials used*

| Material | Viscosity | | Mol Wt |
|---|---|---|---|
| Ethocel | 90-110 | cp | NA |
| Aquazol 50 | 5-7 | cst | 50,000 |
| Aquazol 200 | 18-24 | cst | 200,000 |
| Aquazol 500 | 60-80 | cst | 500,000 |
| Polyox | 1650-5,500 | cp | 4,000,000 |
| PVP | 2.5 | MPa-s | 58,000 |

A polyurethane TPE such as Pellethane is generally referred to as a polyether based thermoplastic polyurethane elastomer ranging from hard to soft and can be fabricated by a variety of methods like injection molding, extrusion and blow molding. These elastomers offer a combination of properties rarely seen in an engineering thermoplastic. It has excellent hydrolytic stability, resistance to fungus and microorganisms. It is prominently used for making catheters, tubings, drug delivery systems, etc.

A polyamide TPE such as Pebax resin belongs to the 33 series from Atofina made up of polyether-block-copolyamide copolymers. The block types and ratios can be varied to achieve a wide range of physical and mechanical properties. Basically Pebax is generally considered a thermoplastic elastomer or a flexible polyamide which consists of a regular linear chain of rigid polyamide segments and flexible polyether segments. It is widely used for medical applications because of its outstanding mechanical properties and good flexibility.

Nylon 12 such as Vestamid is generally considered a thermoplastic material. It is manufactured from the monomer laurolactam. It has one of the lower amide group concentrations among all polyamides that are commercially available. It also may have a low water absorption, excellent impact strength, good dimensional stability and mechanical properties. Due to this combination it is widely used for making dilation catheters, tubings, sporting goods and mechanical applications.

A polyolefin such as Marlex may be a high density ethylene-hexene copolymer and is generally considered to have good toughness, processability and mechanical properties. It is known to have low coefficient of friction.

A very few polymers were available which were hydrophilic in nature, as well as being FDA approved. Ethyl cellulose, polyethylene glycol, polyvinyl pyrrolidone and polyethyl oxazoline were selected based on their solubility in water and their availability in the market.

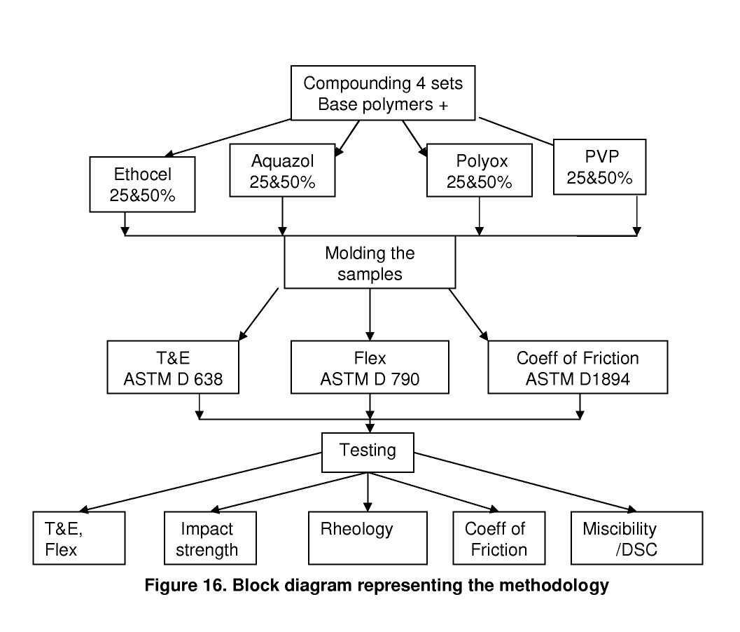

FIG. 16 shows a block diagram representing the methodology followed for the examples described herein. It gives step by step information on how formulation of the examples and testing was carried out.

Melt blending was done on a WP ZSK 30 mm co-rotating twin screw extruder. The specifications for the extruder are given in Table 4. The temperature profile used for each base polymer is given in Table 5. In most cases use of water bath for cooling the strand was avoided due to the presence of hydrophilic polymers. A pelletizer was used to pelletize the strand. A general procedure for melt blending is outlined below.

Pellethane, Pebax and Vestamid were dried in a desiccant dryer for a minimum of 4 hrs at 180° F. prior to batching. Marlex and the hydrophilic polymers were used without drying

A loading level of 25 and 50% was used for each hydrophilic polymer being added to the base polymer. A batch size of 10 lb was made for each loading level. The two polymers being used were weighed and dry blended in a tumbler for 5 min.

The dry blended batch was fed to a single screw feeder placed near the hopper of a twin screw extruder. Screw speed for the feeder was set such that a minimum torque level of 50% was achieved during melt blending on the extruder so as to achieve uniform mixing.

TABLE 4
Extruder Specifications

| Manufacturer | Werner & |
|---|---|
| | Pfleiderer |
| Model | ZSK 30 |
| Type | Co-rotating |
| L/D ratio | 24/1 |

TABLE 5
Temperature Profile used for Compounding Different Base Polymers

| | Zone | Zone | Zone | Zone | Die |
|---|---|---|---|---|---|
| Material | 1 (° C.) | 2 (° C.) | 3 (° C.) | 4 (° C.) | (° C.) |
| Pellethane | 190 | 200 | 210 | 210 | 210 |
| Pebax | 170 | 180 | 185 | 180 | 180 |
| Marlex | 200 | 225 | 220 | 220 | 220 |
| Vestamid | 180 | 210 | 220 | 220 | 210 |

Samples were injection molded in an ASTM test mold to make the tensile and flex bars. The specifications of the injection molding machine are shown in Table 6. A general temperature profile is shown in Table 7.

TABLE 6
Injection Molding Machine Specifications

| | |
|---|---|
| Manufacturer | Arburg Inc. |
| Model | 221 M-350 |
| Clamp force | 350 KN |
| Injection speed | 4.9-95.5 ft/min |
| Temperature | 86-734° C. |
| range | |
| Shot size range | 0-2.9 in³. |

TABLE 7
Temperature Profile used for Molding Different Base Polymers

| | Feed | | | | |
|---|---|---|---|---|---|
| | Zone | Zone | Zone | Zone | Nozzle |
| Material | (° C.) | 1 (° C.) | 2 (° C.) | 3 (° C.) | (° C.) |
| Pellethane | 195 | 195 | 200 | 205 | 205 |
| Pebax | 195 | 205 | 205 | 205 | 205 |
| Marlex | 195. | 195 | 205 | 210 | 210 |
| Vestamid | 230 | 240 | 245 | 250 | 250 |

Tensile testing was performed on the samples using ASTM Test Method D 638. This test is used for screening the materials, selection and quality control purposes. Test results give tensile properties like elongation at yield and break, yield strength, break strength and tensile modulus. This test in a broad sense measures the ability of the material to withstand forces that tend to pull it apart, and determines to what extent the material stretches before breaking. Universal testing machine was used (Model# QT/25 from MTS). Test works version 3.1 was used to analyze the data. Injection molded test specimens of type IV were used. The specimen had a width of 0.125 in. and a thickness of 0.060 in. The distance between the grips was 1 in. and the gauge length used was 1 in. The specimens were conditioned at room temperature for a minimum of 40 hr before testing. Testing was done in standard laboratory atmosphere. The test specimen was placed in the grips of the movable and fixed members. The specimen was adjusted symmetrically so as to distribute the tension uniformly over the cross section. Tensile load was applied at a constant rate of speed of 5 in/min and properties like tensile modulus, elongation at yield and break, yield stress and break stress were recorded. A minimum of five samples were tested for each material.

Flexural properties were tested according to ASTM Test Method D790. This test is generally understood to measure the flexural modulus or stiffness of different materials under load. Flexural strength is also characterized as measuring the ability of the material to withstand bending forces applied perpendicular to its longitudinal axis. It relatively measures the stiffness of each material correlating to the outside surface of the samples. This property is calculated based on the maximum stress and strain that occurs at the surface of the sample. For samples that do not break, results are calculated when the strain in the outer fiber has reached five percent. Universal testing machine was used (Model# QT/25 from MTS). Test works version 3.1 was used to analyze the data. Injection molded test specimens were used with the dimensions of 5 by ½ by ⅛ in. tested flat wise on a support span of 16:1 (span to depth ratio). The span length used was 4 in. The specimens were conditioned at room temperature for a minimum of 40 hr before testing. Testing was done in standard laboratory atmosphere. The sample was placed such that a three point load could be applied on it. The test was initiated by applying a central load to the specimen at a cross head rate of 0.2 in/min. Modulus was calculated when the strain in the outer fibers of the specimen reached five percent. A minimum of five samples were tested for each material.

Charpy Impact Test, ASTM test method D 6110 was performed on the samples. This test is generally considered to determine the impact resistance of materials which is directly related to the toughness and characterizes the ability of the material to resist breaking under a shock load. Molecular flexibility determines whether the material is brittle or tough. Flexible materials have high impact strength due to their ability to respond rapidly to mechanical load. Injection molded flex bars were used for testing impact resistance. The specimen had dimensions of 5 by ½ by ⅛ in. The specimen used was notched to provide a stress concentration point which promotes brittle failure rather than ductile failure. Testing was done in standard laboratory atmosphere. After notching, the specimens were conditioned for a minimum of 48 hrs at standard laboratory conditions to relieve the stress. The specimen was supported horizontally as a simple beam with the notch facing the opposite edge of the striking pendulum. A pendulum type hammer (2-10 lb) was used for striking the specimen so as to see a break. The pendulum was connected to a pointer and dial mechanism that indicated the excess energy remaining in the pendulum after breaking the specimen. Charpy impact strength was calculated by dividing the pointer reading on the dial by the thickness of the sample. For samples that did not break, NB was reported.

Capillary rheometry was performed using ASTM D 1703. Capillary rheometer is generally characterized as measuring the apparent viscosity of the material at different shear rates. The rheometer consists of an electrically heated cylinder, temperature controlling unit and a piston which moves at a constant velocity. Velocity correlates to the shear rate and the force required in moving the piston determines the shear stress. (Model # LCR 7000 from Dynisco was used). A few grams of sample were used for each test. Samples containing Pebax, Pellethane and Vestamid were dried for 4 hrs at 180° F. in a vacuum dryer prior to testing. The barrel was heated to a constant temperature based on the material. The sample was loaded in to the barrel, preheated for 360 seconds and then forced out of the die by the piston at a predetermined shear rate. Preheating helped in melting the sample uniformly before testing. A die with LID ratio of 15:1 was used. Pellethane was tested at 224° C., Marlex were tested at 190° C. while Vestamid and Pebax were tested at 235° C.

The coefficient of friction was determined using ASTM Test Method D 1894. This test method is generally characterized as measuring the coefficients of starting and sliding friction of a plastic when sliding over itself or any other substances at specified test conditions. Universal testing machine was used. (Model# QT/25: MTS). A sled was attached to the load cell and the sample to be tested was placed on a supporting base. Test works version 3.1 was used to analyze the data. A 5 in. by 5 in. sample was used and was placed on the supporting base. Thickness of the sample was ⅛ in. The specimens were conditioned at room temperature for a minimum of 40 hrs before testing. The sample was taped on the supporting base and was tested against a stainless steel surface. A load cell of 5 lb was used to pull the sled against the specimen surface at a specified cross head rate and the results are recorded.

Differential Scanning Calorimetry was performed on the samples. DSC is a thermal analysis technique which is generally characterized as determining the melting behavior, melting point, glass transition temperature, degree of cross linking and degradation behavior of various polymers. ASTM Method D-3417 was used. 10 to 20 mg of sample was used to test the samples. TA instruments model number 2920 was used to test the samples. The sample was weighed and placed in an aluminum pan along with a reference pan which was empty. Both the pans were heated at a constant rate of 10° C./min in an inert atmosphere. The change in energy required to heat the samples was plotted against temperature to get different results like glass transition temperature and melting pint.

Different materials were evaluated after melt blending them at concentrations of 25 and 50% and results obtained are discussed in the following chapters. Results have been divided based on different base materials used. In each section the effect of blending ratio on the mechanical properties like tensile strength, yield strength, elongation at yield, flexural strength, impact strength, viscosity, coefficient of friction and thermal properties has been analyzed.

Among the base polymers, based on the hydrogen bonding, it can be said that Pellethane, Vestamid and Pebax are polar in nature while Marlex which is ethylene based is non-polar in nature. For the hydrophilic polymers, based on the chemistry, these polymers can be ranked in the order as PVP being highly polar in nature, followed by Ethocel and Aquazol. Polyox is the least polar among these hydrophilic polymers. This can be correlated to some extent to the compatibility of the blends. Actual solubility parameter numbers and hydrogen bonding values would be required to make conclusions about miscibility for all the blends.

To achieve a low coefficient of friction Pellethane was melt blended with hydrophilic polymers and its final properties were evaluated. Table 8 gives comparative data for the different blends seen relative to virgin Pellethane. It can be seen that PVP, Aquazol and Ethocel at 25% loading reduced both static and dynamic coefficient of friction drastically. The effect of these materials on the mechanical properties was further evaluated. Due to problems in molding, like shrinkage and incompatibility of 25% Polyox with Pellethane, results for this blend have been omitted during the discussion.

TABLE 8
Comparative Data for Pellethane and its Blends

| | Break | % | Tensile | Flex | | | Viscosity@ | Impact |
|---|---|---|---|---|---|---|---|---|
| | stress | Strain @ | modulus | Modulus | Static | Dynamic | 100/s | strength |
| Material | (MPa) | Break | (MPa) | (MPa) | COF | COF | (Pa-s) | (J/m) |
| Pellethane | 46 | 974 | 12 | 109 | 0.78 | 0.82 | 309 | NB |
| virgin | | | | | | | | |
| Pellethane + 25% | 26 | 162 | 394 | 465 | 0.02 | 0.03 | 461 | NB |
| PVP | | | | | | | | |
| Pellethane + 50% | 29 | 6 | 1276 | 1510 | 0.02 | 0.02 | 554 | 20.29 |
| PVP | | | | | | | | |
| Pellethane + 25% | 41 | 491 | 115 | 154 | 0.17 | 0.09 | 155 | NB |
| Aquazol | | | | | | | | |
| Pellethane + 50% | 33 | 166 | 690 | 637 | 0.06 | 0.03 | 170 | 28.19 |
| Aquazol | | | | | | | | |
| Pellethane + 50% | 8 | 284 | 73 | 174 | 0.15 | 0.12 | 1308 | NB |
| Polyox | | | | | | | | |
| Pellethane + 25% | 31 | 182 | 262 | 239 | 0.25 | 0.1 | 702 | NB |
| Ethocel | | | | | | | | |
| Pellethane + 50% | 63 | 12 | 1341 | 932 | 0.08 | 0.06 | 1587 | 81.15 |
| Ethocel | | | | | | | | |

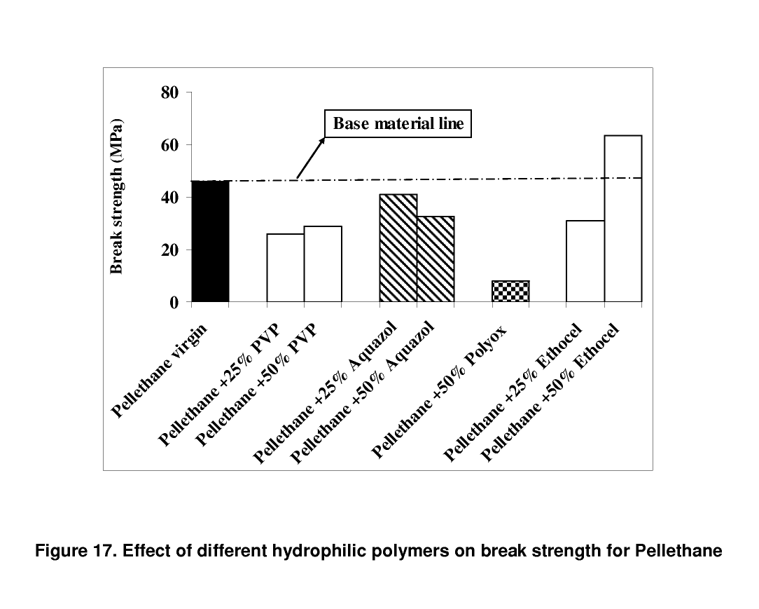

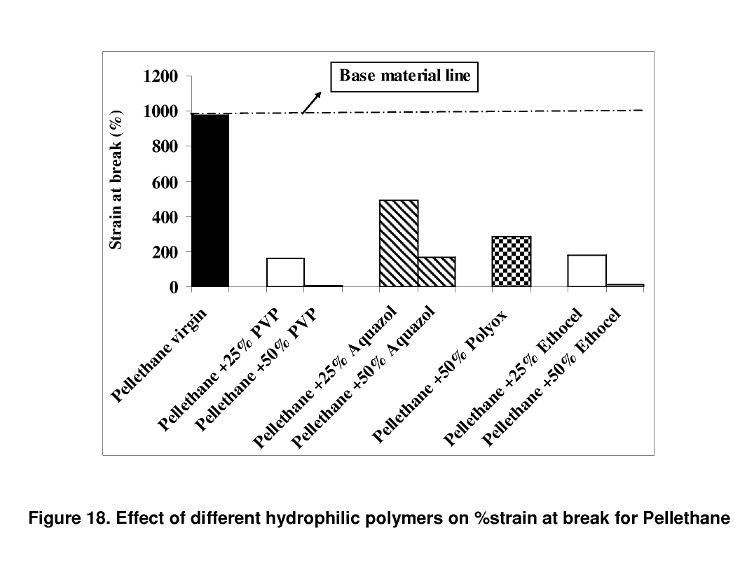

FIGS. 17 and 18 show the effect of different hydrophilic polymers on break strength and elongation at break. It can be seen that except for Ethocel at 50% loading, break strength was reduced considerably for the other blends. Break strength indicates the ability of the material to withstand forces trying to break it apart. Inherently Pellethane has good break strength along with high elongation, but addition of different hydrophilic polymers to it reduced these properties. It was also observed that Pellethane did not yield; hence break strength was used to analyze the data. With the exception of Ethocel at 50% loading, break strength for the blends was reduced. Polyox being non polar in nature, its compatibility with Pellethane was very low. This can be seen from the physical properties data and visual observation during compounding where it resulted in phase separation during compounding and molding. Pellethane is known for its high elongation; however, upon addition of different polymers it can be seen that elongation was reduced drastically. This indicates that these blends would not be as flexible as the virgin Pellethane. The hydrophilic polymers being used were stiff in nature due to their chemical structure and this resulted in the reduction in elongation of Pellethane.

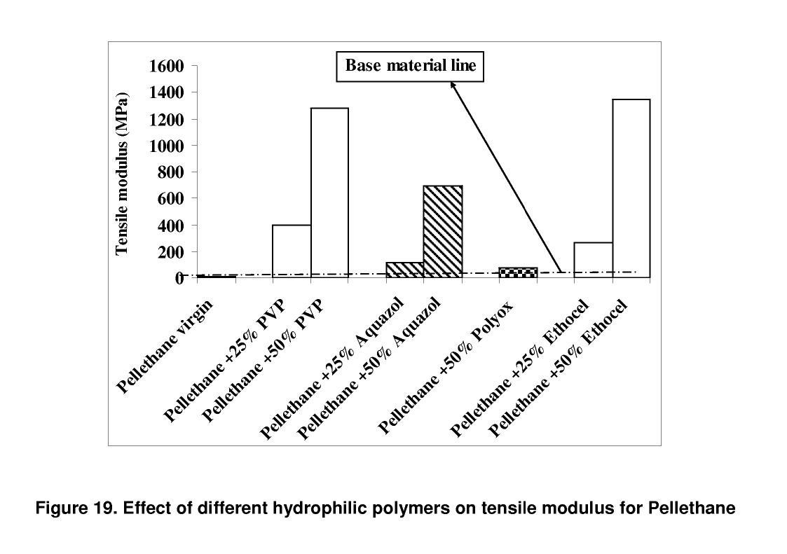

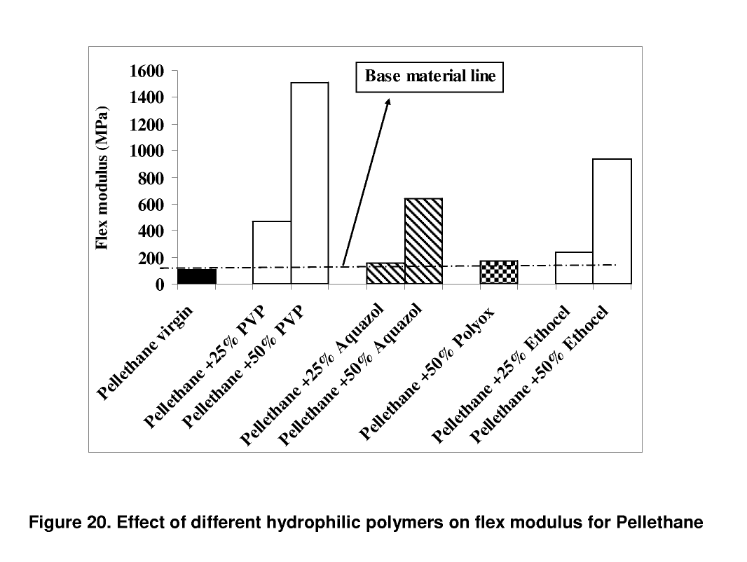

FIGS. 19 and 20 show the effect of different polymers on tensile modulus and flexural modulus for Pellethane. Usually blends follow an additive rule for tensile modulus and flex modulus. Results showed an increase in these properties with the loading level of hydrophilic polymers. This indicated that the hydrophilic polymers being considered were more stiff than Pellethane and led to an overall increase in the modulus. Polyvinyl pyrrolidone and Ethocel show the greatest effect on these properties. It can also be seen that a loading level of 50% showed greater effect on these properties than when compared to 25% loading. However, at higher loadings, elongation at break was reduced drastically, thus limiting the loading level to less than 25%.

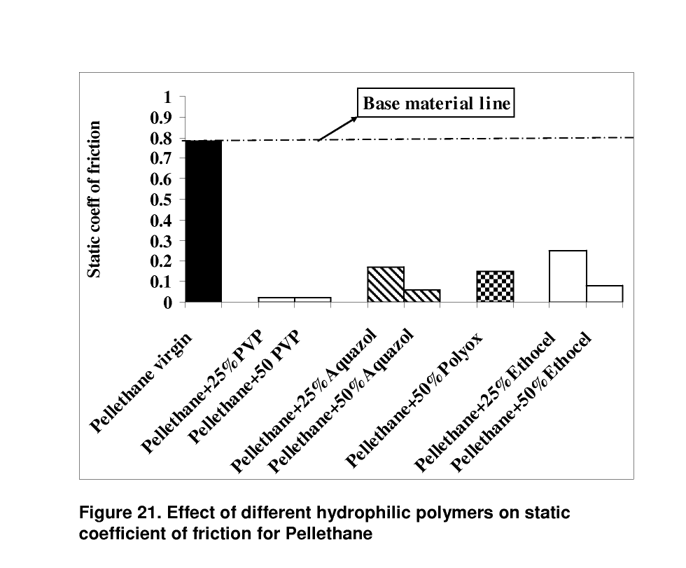

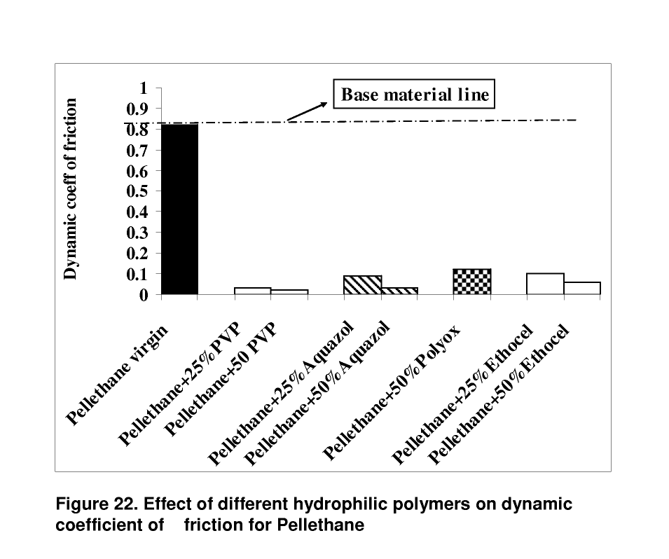

It can be seen from FIGS. 21 and 22 that Pellethane in its virgin form had high static and dynamic coefficient of friction. This was one of the most important problem associated with Pellethane and limited its use in the medical industry. It is highly tacky in nature, making the surfaces stick to itself and other surfaces. Different blends showed that addition of hydrophilic polymers reduced both static and dynamic coefficient of friction. It was observed that the surface became smooth in most cases. It was difficult to determine the wet friction for these materials as there was no standard to measure it.

Evaluation of different materials with Pellethane showed that polyvinyl pyrrolidone and Aquazol reduced friction to a greater extent when compared to Ethocel and Polyox. Overall, data showed that coefficient of friction for Pellethane could be reduced by the addition of hydrophilic polymers. A prediction can be made that these blends will have good lubricity when in contact with water, because of the presence of hydrophilic polymers. A further evaluation would be required to study this property in the wet state. Visual observations during compounding showed that the surface of the strands for all materials became slippery when treated with water. This was a positive sign and would require supporting data to prove that these hydrophilic polymers would reduce surface roughness when in contact with water. In case of polyvinyl pyrrolidone coefficient of friction was similar at both loading levels of 25 and 50% respectively. This indicated that a loading level of 25% would be enough to reduce the coefficient of friction for Pellethane and is worth evaluating further.

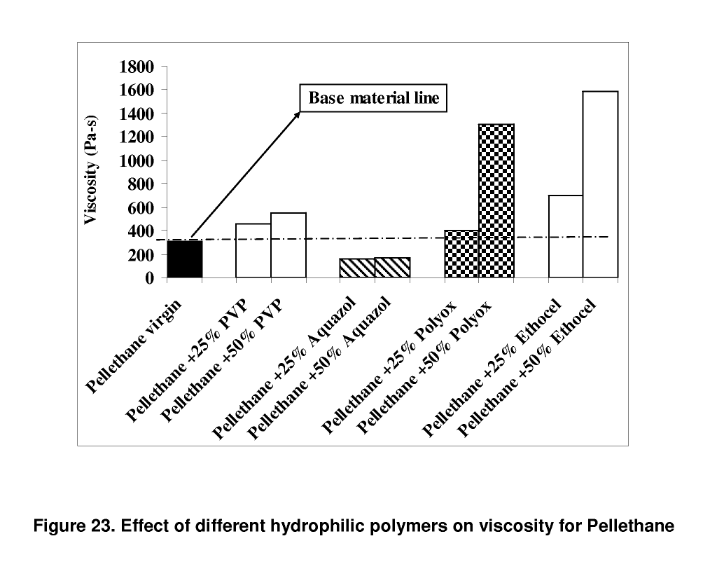

Viscosity of a polymer blend is dependent on the molecular weight, molecular structure, the melting point of individual components and the blend ratio. The grade of Pellethane selected had a hardness value of 80 Shore A and was soft in nature. It can be seen from FIG. 23 that addition of Ethocel, PVP and Polyox increased the viscosity of Pellethane while Aquazol reduced the viscosity. The Aquazol being used was a mixture of low and high molecular weight grades. Low molecular weight component might have acted as a plasticizer and hence reduced the viscosity for Pellethane. Polyox and Ethocel at 50% loading increased the viscosity drastically.

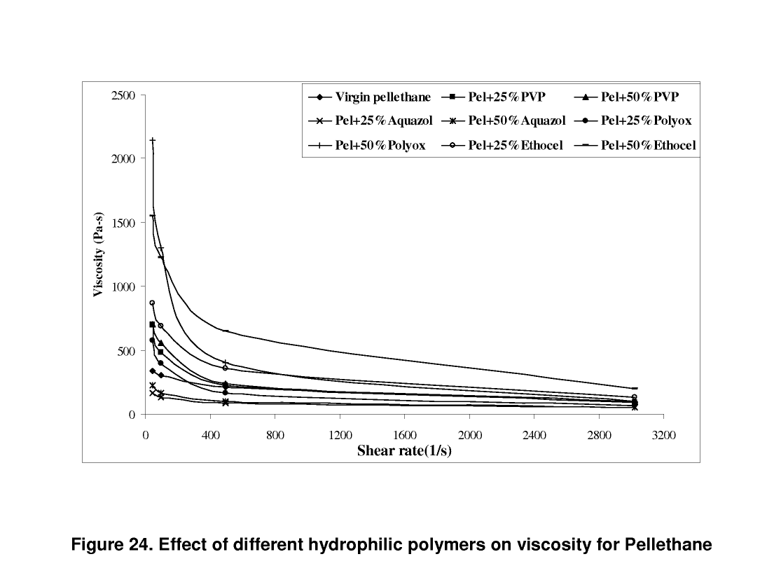

FIG. 24 shows the plot of viscosity vs. shear rate for the different blends with Pellethane. It can be seen that these blends were shear sensitive in the low shear rate region. At very high shear rates, these blends showed very little change in viscosity.

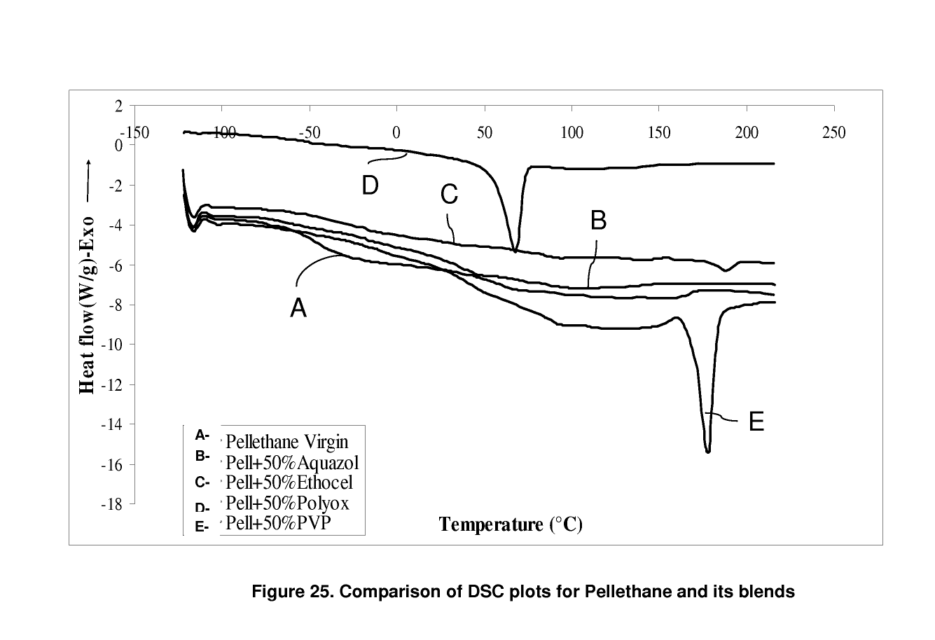

DSC plot for Pellethane showed a Tg of −37° C. which was close to what the manufacturer specified. It is amorphous in nature and behaves like rubber. Comparison of Pellethane and its blends is shown in FIG. 25. It can be seen that, with the exception of Polyox, all other blends appeared miscible since none of the plots showed two individual Tg's corresponding to the components. Pellethane being polar in nature would show some miscibility with the hydrophilic polymers because of the presence of hydrogen bonding on these polymers. It was difficult to distinguish between Tg for Polyox and Pellethane and hence miscibility could not be studied.

Addition of PVP and Polyox showed melting peaks indicating that these polymers formed a crystalline phase in Pellethane, which was inherently amorphous. Table 9 shows the crystallinity data for Pellethane and its blends which indicate that Ethocel, PVP and Polyox formed a crystalline phase. The crystalline phase formed contributed to higher stiffness for these blends.

TABLE 9
Effect of Hydrophilic Polymers on the Physical State for Pellethane

| | | Melting |
|---|---|---|
| Polymer | Physical state | point ° C. |
| Pellethane | Amorphous | — |
| Pellethane + 50% | Amorphous | — |
| Aquazol | | |
| Pellethane + 50% | 2.35% Crystallized | 188.57 |
| Ethocel | | |
| Pellethane + 50% Polyox | 24.67% Crystallized | 67.57 |
| Pellethane + 50% PVP | 34.23% Crystallized | 178.74 |

Pellethane in its virgin form had excellent impact strength. Table 10 shows the results for impact strength of Pellethane and its blends. It can be seen that virgin Pellethane did not break nor did some other blends, indicating that they had good impact strength. However PVP, Aquazol and Ethocel broke at 50% loading indicating that at such high loadings Pellethane lost its flexibility and its impact strength was reduced drastically. Addition of Polyox to Pellethane did not affect the impact strength as results showed that the samples did not break even at higher loading levels.

TABLE 10
Effect of Hydrophilic Polymers on Impact Strength for Pellethane

| | Impact strength |
|---|---|
| Material | (J/m) |
| Pellethane virgin | NB |
| Pellethane + 25% PVP | NB |
| Pellethane + 50% PVP | 20 |
| Pellethane + 25% Aquazol | NB |
| Pellethane + 50% Aquazol | 28 |
| Pellethane + 25% Polyox | NB |
| Pellethane + 50% Polyox | NB |
| Pellethane + 25% Ethocel | NB |
| Pellethane + 50% Ethocel | 81 |

Pebax belongs to the family of nylon based thermoplastic elastomers. The grade selected was the softest, with a hardness of 25 Shore D (75 Shore A). When compared to Pellethane it can be seen that Pebax had higher coefficient of friction and was more tacky in nature. This was the most ideal material to be evaluated to see the effect of different hydrophilic polymers on the coefficient of friction. It can be seen that at low loading level of 25% PVP was the most successful material in reducing the coefficient of friction, while at 50% loading most materials reduced this value. After achieving the primary objective of this project, these blends were later evaluated for mechanical properties. Table 11 summarizes the properties seen for all the blends with Pebax. As mentioned with Pellethane, Pebax is polar in nature due to the presence of hydrogen bonding in its backbone and would have some miscibility with the hydrophilic polymers, which were polar in nature to some extent.

TABLE 11
Comparative Data for Pebax and its Blends

| | Break | % | Tensile | Flex | | | Viscosity@ | Impact |
|---|---|---|---|---|---|---|---|---|
| | stress | Strain @ | Modulus | Modulus | Static | Dynamic | 100/s | strength |
| Material | (MPa) | Break | (MPa) | (MPa) | COF | COF | (Pa-s) | (J/m) |
| Pebax | 21 | 625 | 16 | 28 | 4.35 | 3.05 | 244 | NB |
| Virgin | | | | | | | | |
| Pebax + 25% | 18 | 687 | 18 | 53 | 0.39 | 0.36 | 412 | NB |
| PVP | | | | | | | | |
| Pebax + 50% | 19 | 5 | 602 | 544 | 0.1 | 0.08 | 1019 | 17.94 |
| PVP | | | | | | | | |
| Pebax + 25% | 13 | 214 | 148 | 146 | 1.05 | 1.1 | 206 | NB |
| Aquazol | | | | | | | | |
| Pebax + 50% | 23 | 5 | 876 | 702 | 0.35 | 0.13 | 166 | 29.90 |
| Aquazol | | | | | | | | |
| Pebax + 25% | 12 | 991 | 10 | 97 | 1.19 | 1.69 | 365 | NB |
| Polyox | | | | | | | | |
| Pebax + 50% | 4 | 426 | 46 | 153 | 0.29 | 0.2 | 1170 | NB |
| Polyox | | | | | | | | |
| Pebax + 25% | 15 | 49 | 367 | 304 | 0.91 | 0.63 | 259 | NB |
| Ethocel | | | | | | | | |
| Pebax + 50% | 51 | 7 | 1466 | 929 | 0.28 | 0.22 | 298 | 164.01 |
| Ethocel | | | | | | | | |

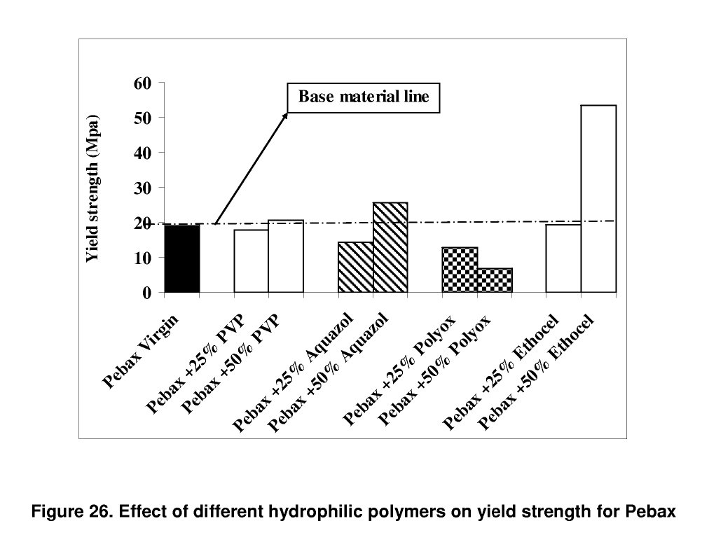

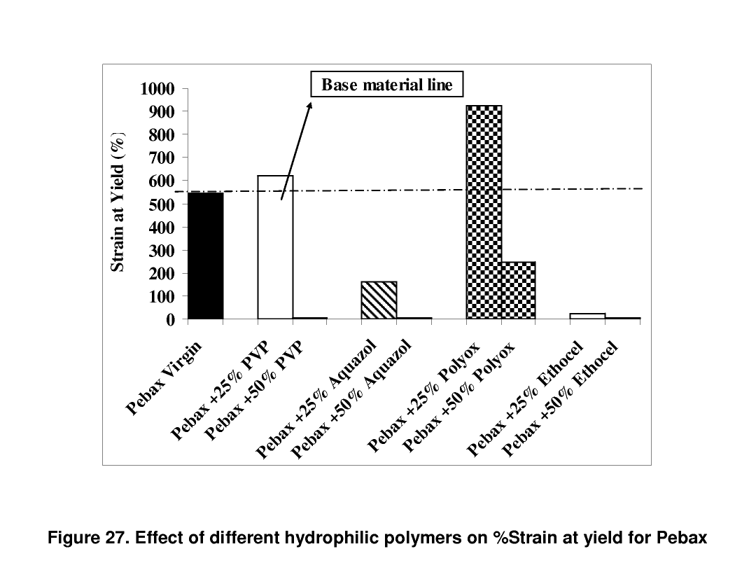

Yield strength represents the stress at which non-elastic deformation occurs, and is important for generating specifications for particular applications like catheters and tubings. Yield strength is useful for applications where force is applied on the material. It gives a limit on the force that can be applied on the material before permanent deformation occurs. It can be seen from FIG. 26 that addition of hydrophilic polymers to Pebax marginally affected the yield strength, with the exception of Polyox which reduced it. FIG. 27 shows that for most blends, elongation at yield was reduced drastically when compared to virgin Pebax. Overall it can be seen that PVP at 25% loading showed good yield strength and elongation at yield, and seemed to be a promising candidate.

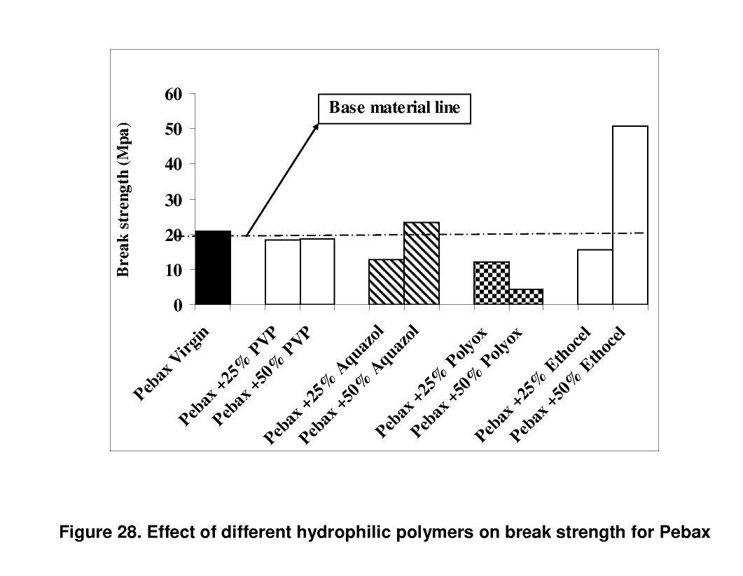

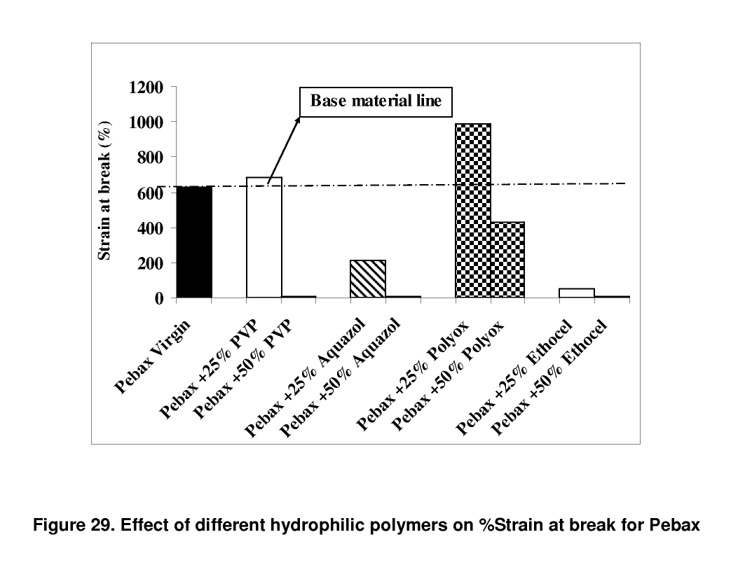

FIGS. 28 and 29 show break strength and elongation at break for Pellethane and its blends. Break strength for blends gives a good idea about the compatibility of the individual components present in the blend. The trend seen was similar to the yield strength seen in these materials. Overall, PVP at 25% loading showed good ultimate break strength and elongation at break.

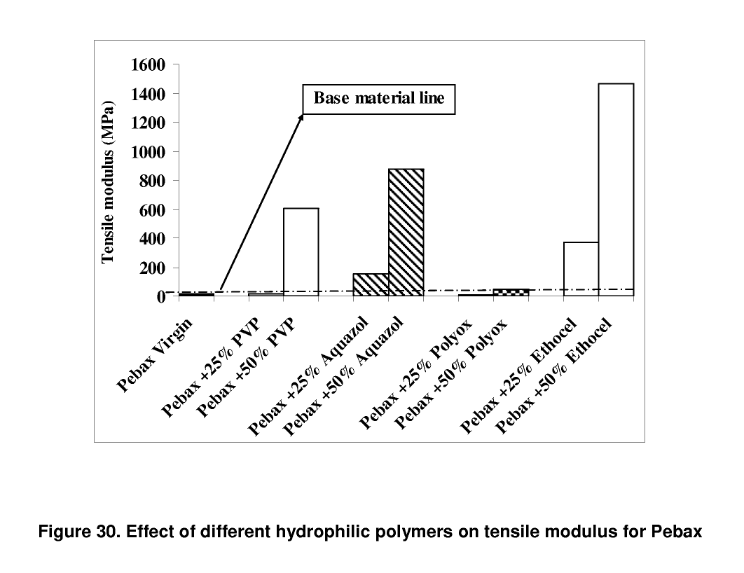

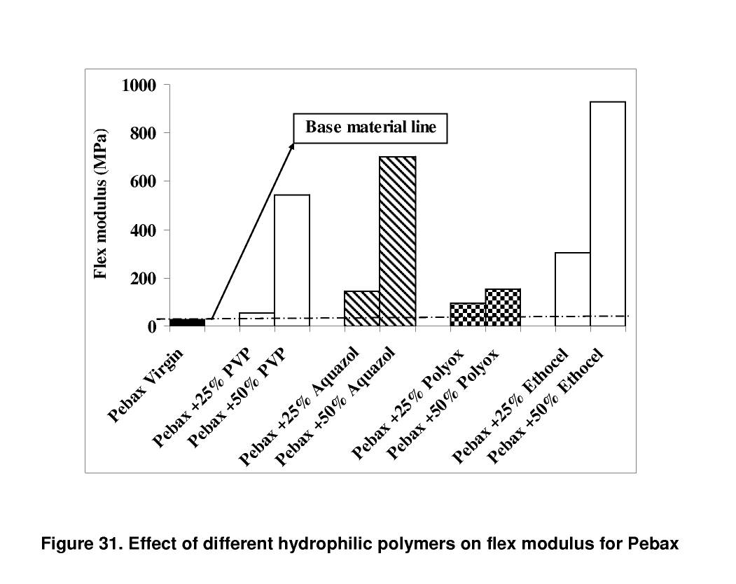

FIGS. 30 and 31 show the effect of different hydrophilic polymers on tensile and flex modulus for Pebax. Addition of Ethocel and Aquazol resulted in an increase in tensile modulus, while flex modulus increased for all blends. A loading level of 50% showed prominent increase in these properties. When considering these blends for different applications the loss in elongation has to be considered at higher loading levels.

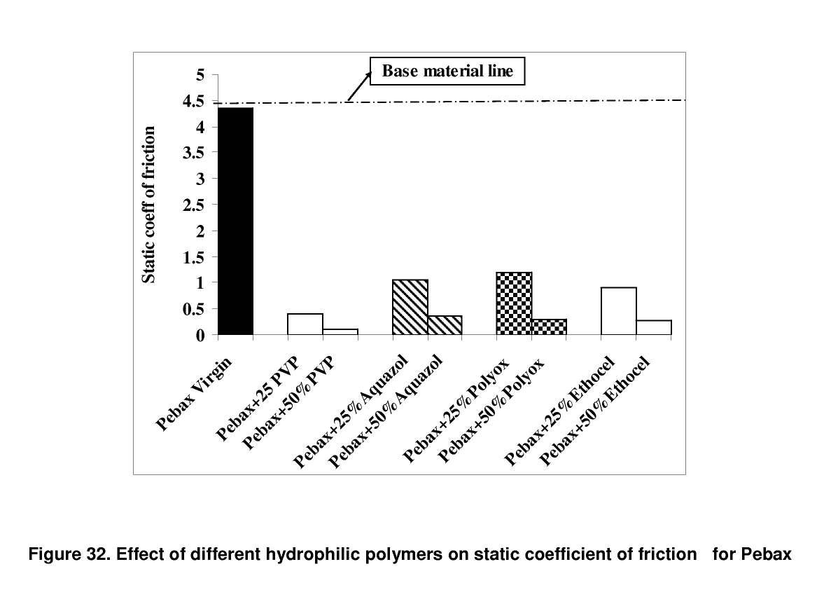

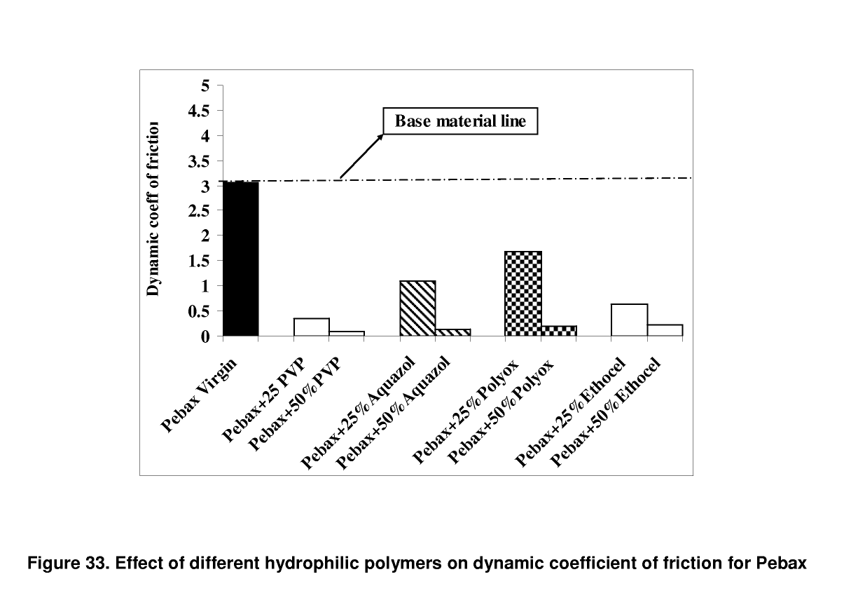

FIGS. 32 and 33 show that Pebax had high static and dynamic coefficient of friction when used alone. Addition of hydrophilic polymers resulted in a decrease in both static and dynamic coefficient of friction. Results were promising even at low loadings, indicating that these blends would have a smoother surface when used for different applications. Use of higher loading level reduced these values even more, but a compromise has to be achieved for different properties when considering it for practical applications. Based on the visual observations for Polyox-Pebax blend it can be said that these polymers were compatible since they formed a phase separated blend.

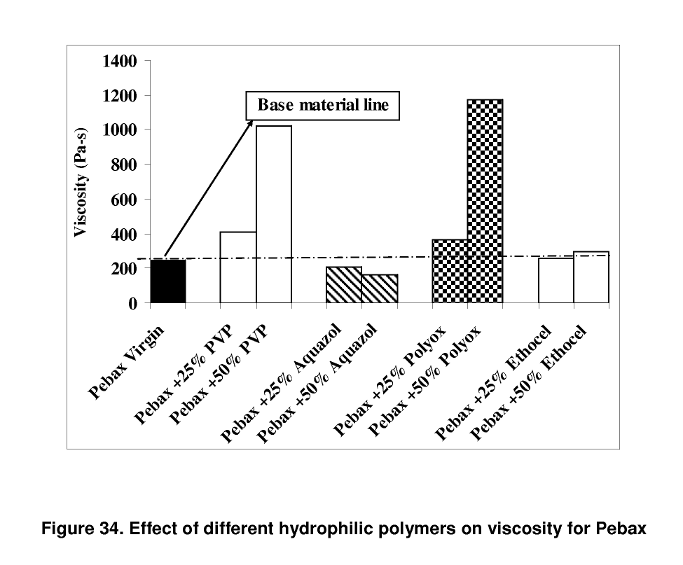

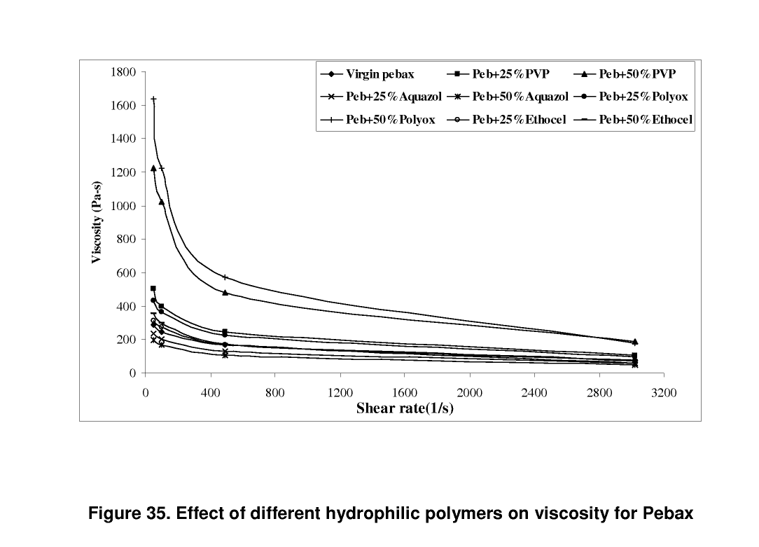

FIG. 34 shows that addition of PVP and Polyox to Pebax increased viscosity, which may be attributed to the stiffness of PVP and the high molecular weight of Polyox. A small reduction in viscosity was seen when Aquazol was added to Pebax, which may have been due to the presence of low molecular weight component in the blend. Not much effect was seen when Ethocel was added to Pebax. FIG. 35 shows the plot for viscosity vs. shear rate which indicates that, as shear rate increased viscosity decreased. All blends were sensitive to shear at low shear rates but the effect was negligible at higher shear rates.

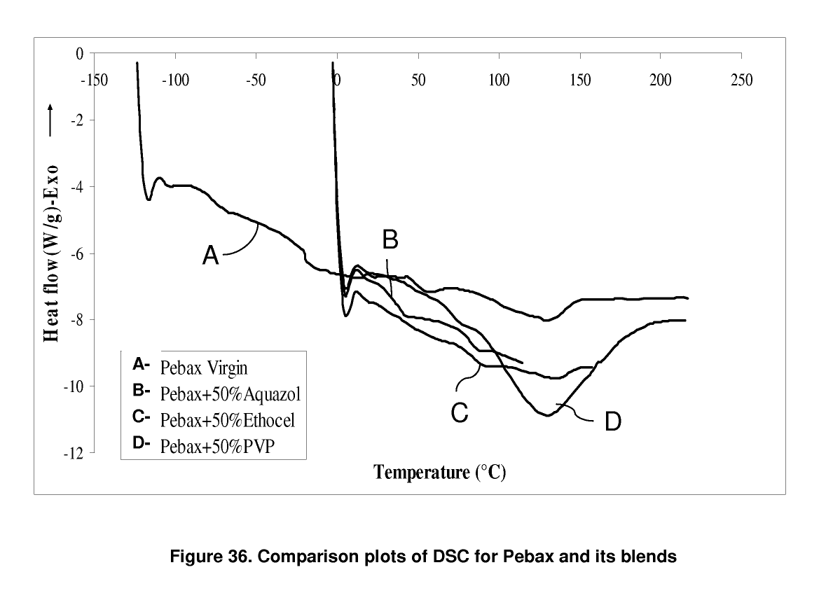

DSC plot for Pebax showed a Tg of −60° C. Comparison plots for different blends are shown in FIG. 36. It can also be seen that Aquazol and Ethocel were miscible with Pebax, as we see a shift in their Tg towards the Tg of Pebax. Plot of PVP showed the formation of a small hard phase which melted around 130° C. Ethocel showed a small peak in the similar temperature range indicating the formation of a crystalline phase. Miscibility of PVP with Pebax could not be analyzed as its Tg was around the vicinity of the melting range for this material. DSC plot for Pebax and Polyox was avoided since the Tg's of these polymers were close to each other.

Like Pellethane, Pebax also had high impact strength and did not break even upon notching. It can be seen from Table 12 that results obtained were similar to Pellethane. PVP, Polyox and Ethocel at 50% loading indicated a break value. This shows that these polymers reduced the impact strength for Pebax at higher loading levels. Polyox did not seem to affect impact strength of Pebax.

TABLE 12
Effect of Hydrophilic Polymers on Impact Strength for Pebax

| | Impact strength |
|---|---|
| Material | (J/m) |
| Pebax Virgin | NB |
| Pebax + 25% PVP | NB |
| Pebax + 50% PVP | 17.94 |
| Pebax + 25% Aquazol | NB |
| Pebax + 50% Aquazol | 29.90 |
| Pebax + 25% Polyox | NB |
| Pebax + 50% Polyox | NB |
| Pebax + 25% Ethocel | NB |
| Pebax + 50% Ethocel | 164.01 |

Vestamid belongs to the family of nylon based thermoplastics but is much stiffer than Pebax. When compared to Pellethane and Pebax it can be seen that Vestamid has low coefficient of friction and is currently being used in many applications requiring lower coefficient of friction. Table 13 summarizes the data obtained for all blends being considered with Vestamid. It can be seen that all materials being used with Vestamid reduced static and dynamic coefficient of friction. Vestamid is polar in nature due to the presence of hydrogen bonding and would show some miscibility with hydrophilic polymers, similar to Pellethane and Pebax.

TABLE 13
Comparative Data for Vestamid and its Blends

| | Break | % | Tensile | Flex | | | Viscosity@ | |
|---|---|---|---|---|---|---|---|---|
| | stress | Strain @ | Modulus | Modulus | Static | Dynamic | 100/s | Impact |
| Material | (MPA) | Break | (MPa) | (MPa) | COF | COF | (Pa-s) | strength (J/m) |
| Vestamid | 65 | 187 | 844 | 1052 | 1.4 | 1.2 | 2109 | 210.14 |
| virgin | | | | | | | | |
| Vestamid + 25% | 63 | 177 | 1159 | 1524 | 0.09 | 0.07 | 2280 | 44.42 |
| PVP | | | | | | | | |
| Vestamid + 50% | 65 | 7 | 1726 | 2012 | 0.06 | 0.03 | 3313 | 29.90 |
| PVP | | | | | | | | |
| Vestamid + 25% | 28 | 145 | 1245 | 1365 | 0.14 | 0.04 | 1002 | 62.36 |
| Aquazol | | | | | | | | |
| Vestamid + 50% | 64 | 6 | 1676 | 1722 | 0.06 | 0.03 | 440 | 38.44 |
| Aquazol | | | | | | | | |
| Vestamid + 25% | 36 | 86 | 880 | 857 | 0.05 | 0.03 | 2006 | 125.57 |
| Polyox | | | | | | | | |
| Vestamid + 50% | 17 | 34 | 872 | 999 | 0.09 | 0.05 | 3057 | 80.30 |
| Polyox | | | | | | | | |
| Vestamid + 25% | 40 | 15 | 1320 | 1800 | 0.07 | 0.05 | 1059 | 138.39 |
| Ethocel | | | | | | | | |
| Vestamid + 50% | 67 | 8 | 1728 | 1511 | 0.16 | 0.08 | 772 | 144.37 |
| Ethocel | | | | | | | | |

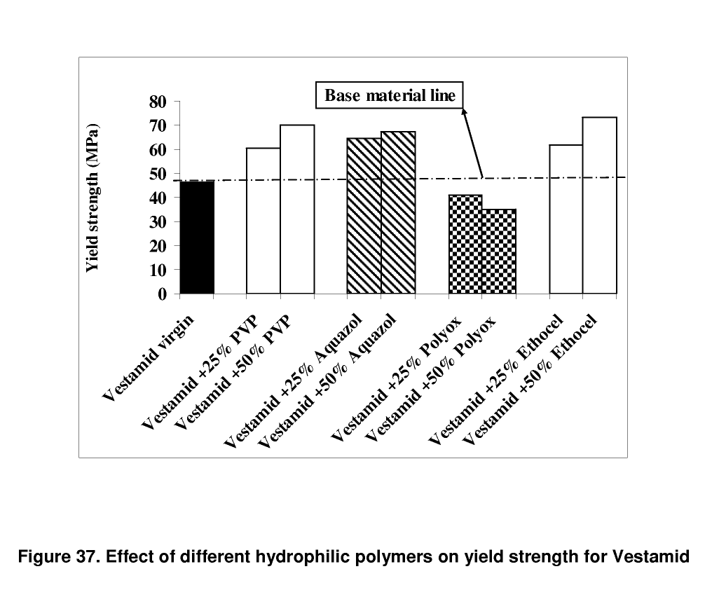

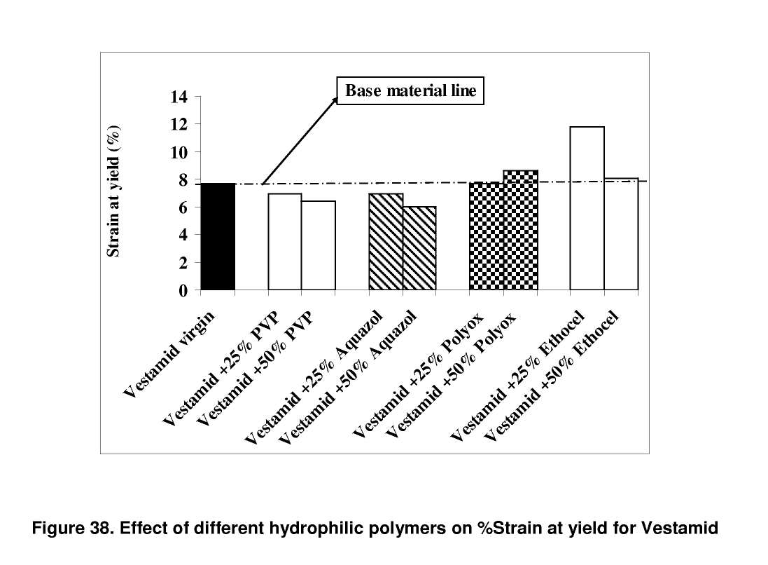

Inherently Vestamid has good mechanical properties; hence the main objective with this resin was to reduce the coefficient of friction without losing much of mechanical properties. FIGS. 37 and 38 show the effect of different materials on yield strength and elongation at yield of Vestamid. It can be seen that, with the exception of Polyox, other materials increased the yield strength of Vestamid. All the blends did not lose much on elongation at yield when compared to the base resin.

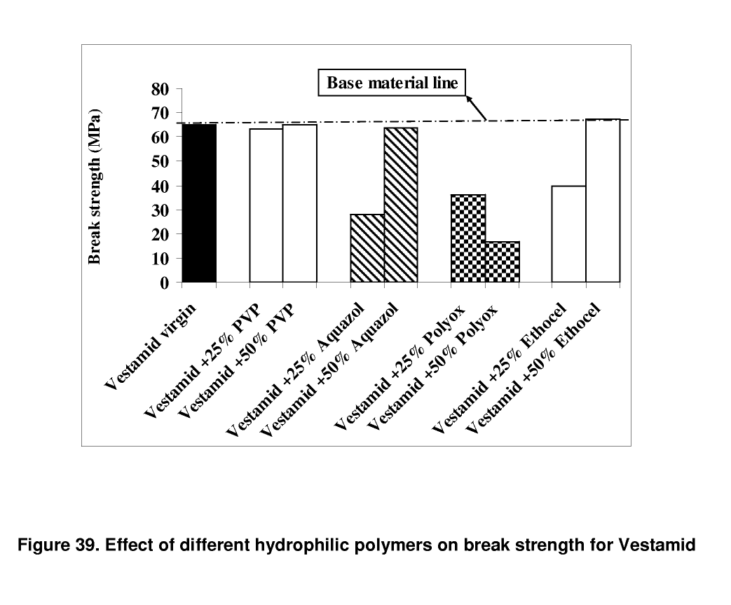

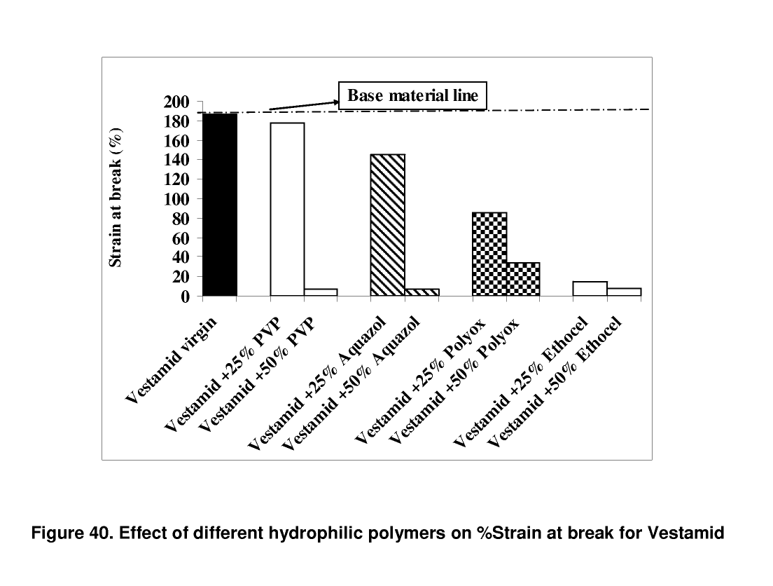

FIGS. 39 and 40 show the results for break strength and elongation at break for Vestamid and its blends. Vestamid is a semi-crystalline material and has good tensile strength along with good elongation properties. Among all the blends being considered, Vestamid with PVP at 25% loading seemed to give the optimum results for tensile strength at break and elongation at break. The loss in elongation seen at 50% loading was quite high and could be due to phase separation between the two polymers being considered. This proved that 50% loading level would not work with Vestamid for any polymer due to the loss in elongation.

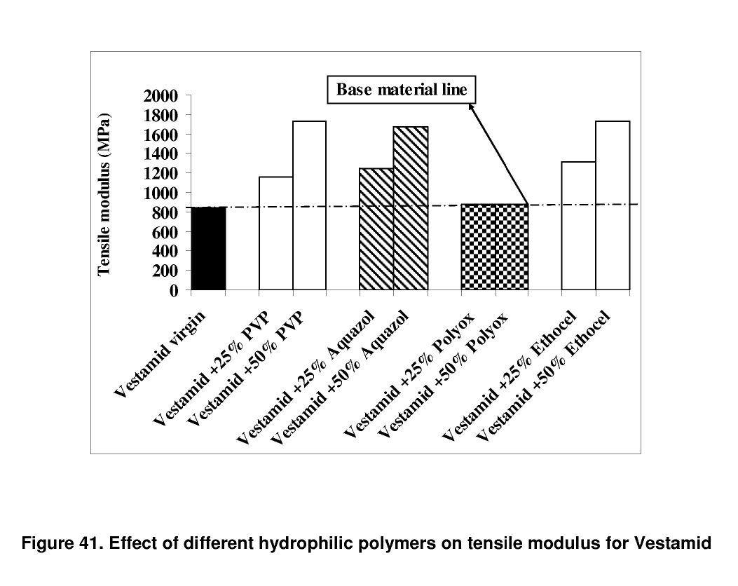

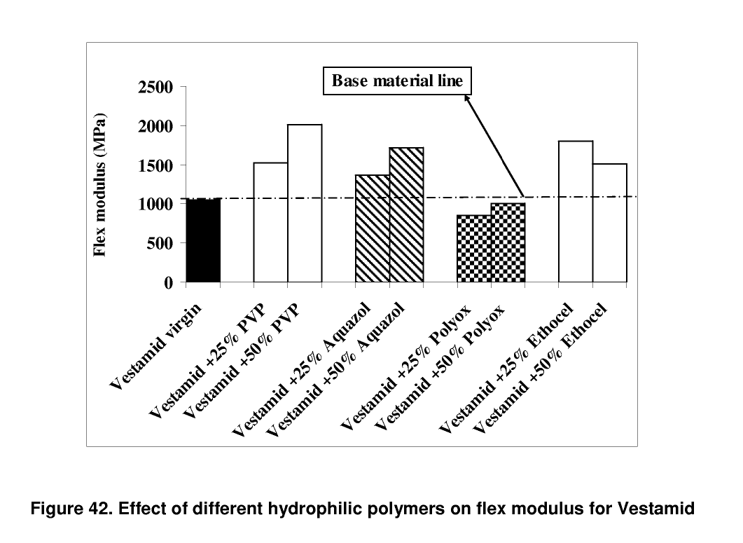

Addition of PVP, Aquazol and Ethocel led to an increase in both tensile and flex modulus when compared to the base resin. There was no effect on tensile modulus when Polyox was added to Vestamid, but the flex modulus decreased. These results are depicted in FIGS. 41 and 42. Overall it can be said that, with the exception of Polyox, other hydrophilic polymers made Vestamid stiffer in nature. Loss in elongation has to be considered before selecting the right blend for final applications.

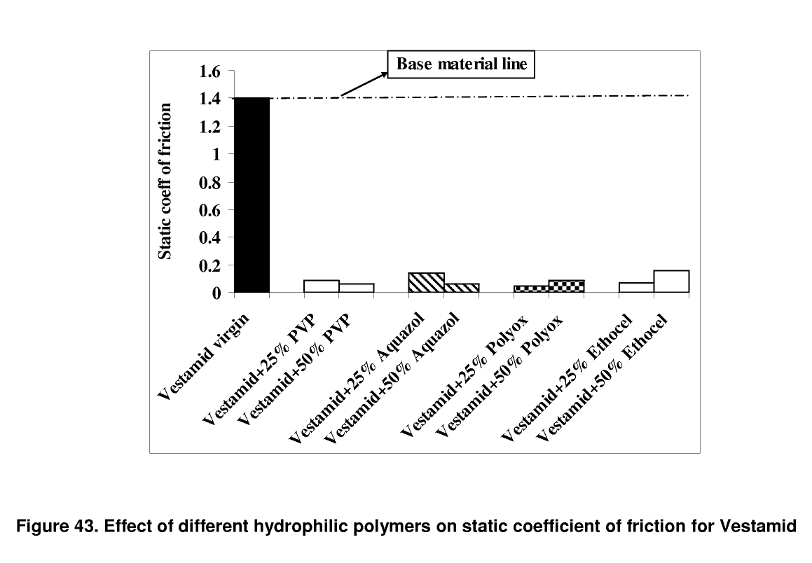

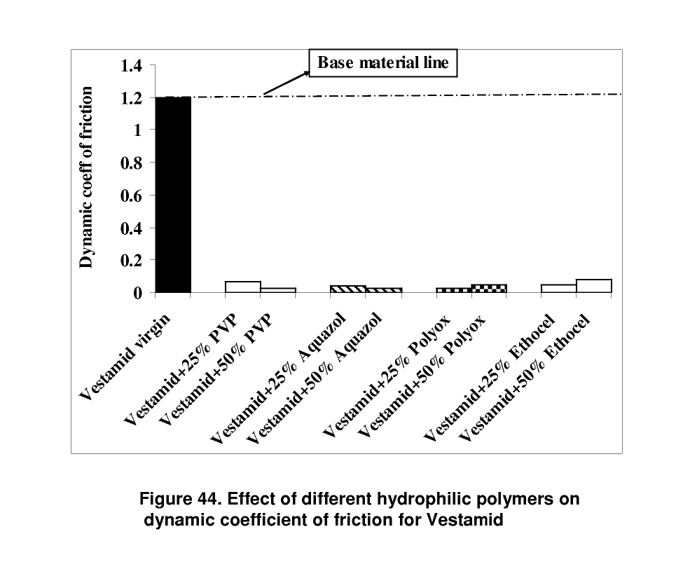

FIGS. 43 and 44 show the plot for static and dynamic coefficient of friction for Vestamid and its blends. It can be seen that all the hydrophilic polymers reduced the coefficient of friction. Based on the combination of properties, PVP at 25% loading and Aquazol at 25% loading gave the best results.

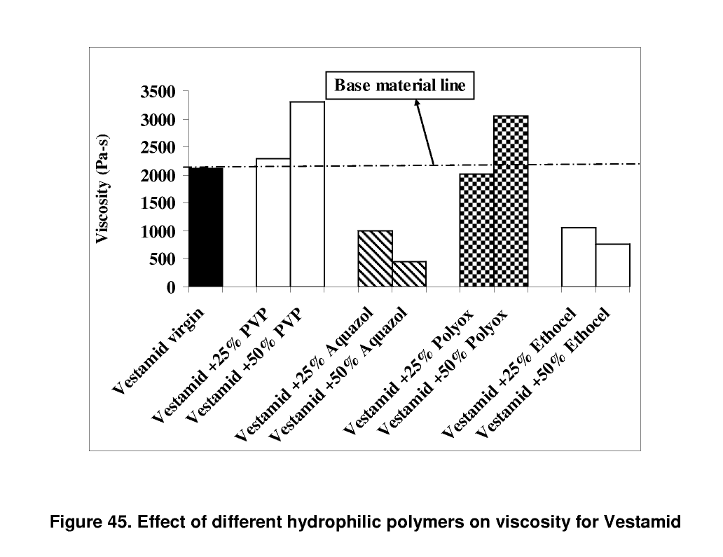

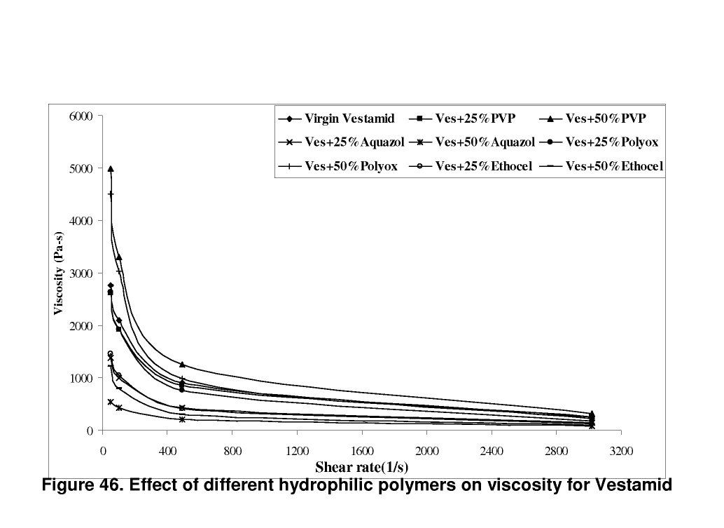

It can be seen from FIG. 45 that addition of Aquazol and Ethocel reduced the viscosity of Vestamid, while PVP and Polyox increased the viscosity at 50% loading without affecting it much at 25% loading. FIG. 46 gives the plot for viscosity vs. shear rate, where it can be seen that the viscosity for the blends was quite different at lower shear rates, but at higher shear rates the change in viscosity was quite small. This indicates that all these blends were sensitive to shear at low shear rates but the effect was negligible at higher shear rates.

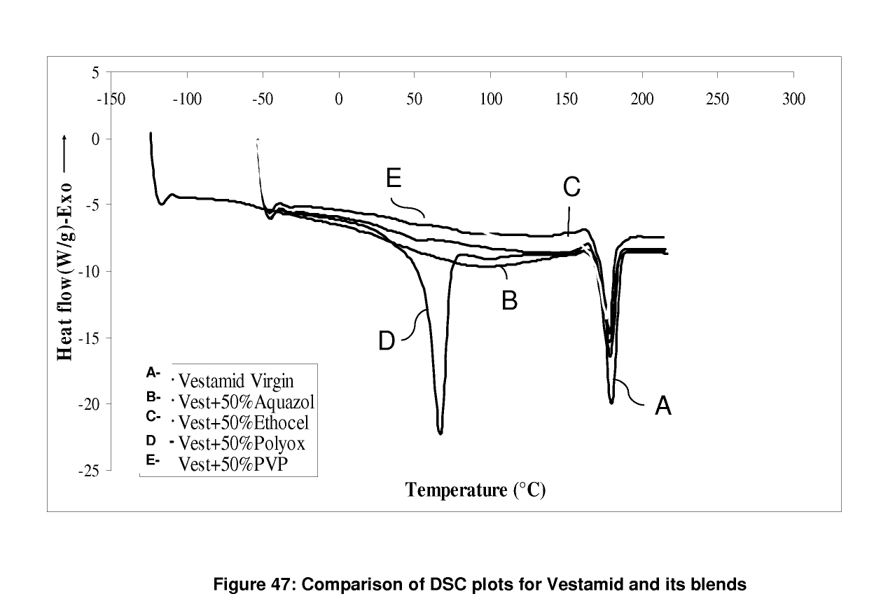

A DSC plot for Vestamid in FIG. 47 showed that it had a Tg around 40° C. and a melting point of 180° C. Addition of different hydrophilic polymers to it showed that the amount of crystallinity was reduced while Polyox showed a second melting peak at around 60° C. It was difficult to judge whether Polyox was miscible with Vestamid because the melting transition seen for Polyox was close to the Tg of Vestamid. Results based on Tg showed that the other polymers being considered were miscible with it.

Table 14 shows the data for effect of different blends on crystallinity for Vestamid. It can be seen that all blends had a melting point around 180° C. It can also be seen that the amount of crystallinity was reduced upon addition of different hydrophilic polymers. In the case of Polyox with Vestamid, two peaks were observed, one for Polyox and the other for Vestamid, indicating two crystalline phases in the polymer matrix.

TABLE 14
Effect of hydrophilic polymers on the physical state for Vestamid

| Polymer | Physical state | Melting point © |
|---|---|---|
| Vestamid | 61.24% Crystallized | 180.3 |
| Vestamid + 50% Aquazol | 29.52% Crystallized | 178.05 |
| Vestamid + 50% Ethocel | 35.2% Crystallized | 178.8 |
| Vestamid + 50% Polyox | 75.94% & 26.8% | 67.59 & 179 |
| | Crystallized | |
| Vestamid + 50% PVP | 31.5% Crystallized | 176.82 |

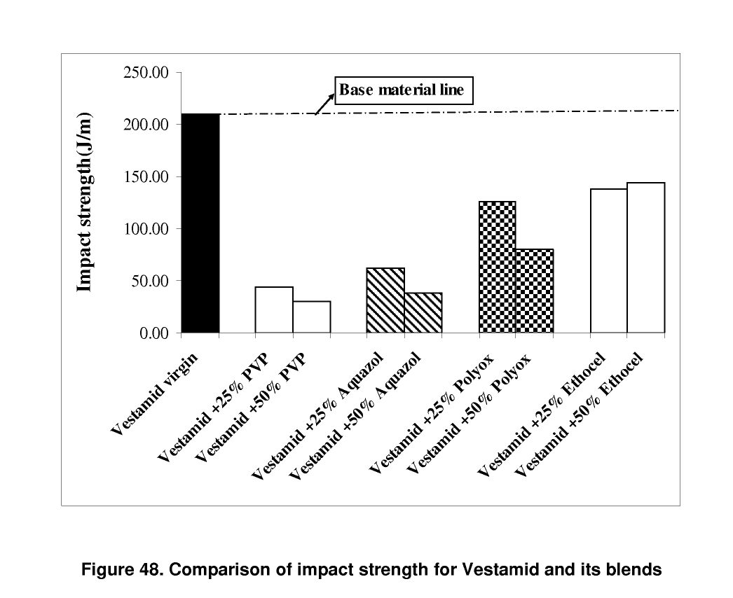

FIG. 48 shows that Vestamid in its virgin form had high impact strength. Inherently Vestamid is a tough material and has good flexibility. However, Table 15 showed that addition of different hydrophilic polymers to Vestamid reduced its impact strength. This showed that these hydrophilic polymers reduced its flexibility and free volume, thus reducing the ability to resist force.

TABLE 15
Effect of hydrophilic polymers on Impact strength for Vestamid

| | Impact strength |
|---|---|
| Material | (J/m) |
| Vestamid virgin | 210.14 |
| Vestamid + 25% PVP | 44.42 |
| Vestamid + 50% PVP | 29.90 |
| Vestamid + 25% Aquazol | 62.36 |
| Vestamid + 50% Aquazol | 38.44 |
| Vestamid + 25% Polyox | 125.57 |
| Vestamid + 50% Polyox | 80.30 |
| Vestamid + 25% Ethocel | 138.39 |
| Vestamid + 50% Ethocel | 144.37 |

Marlex belongs to the family of polyolefin based thermoplastic materials. It is widely used to make inner layers of catheters and sheaths, and is considered to be a tough and versatile material. Polyolefins inherently have low coefficient of friction; hence Marlex was selected so as to evaluate a wide range of base materials with different co-efficient of friction. Marlex in non-polar in nature and will have very little compatibility with hydrophilic polymers selected because of their polarity. Visual observations showed phase separation during compounding and molding, indicating that the blends were not compatible. Table 16 summarizes the data obtained for Marlex and its blends.

It can be seen from the data that none of the hydrophilic polymers used succeeded in reducing the coefficient of friction. The main objective was not achieved in the case of Marlex; evaluation of wet friction would help in understanding whether these hydrophilic polymers would reduce the coefficient of friction in the wet state. A detailed discussion on the different properties for Marlex is limited due to the failure in achieving the main objective.

TABLE 16
Comparative Data for Marlex and its blends

| | Break | % | Tensile | Flex | | | Impact |
|---|---|---|---|---|---|---|---|
| | stress | Strain @ | Modulus | Modulus | Static | Dynamic | strength |
| Material | (MPa) | Break | (MPa) | (MPa) | COF | COF | (J/m) |
| Marlex | 14 | 23 | 1022 | 629 | 0.16 | 0.08 | 269.94 |
| virgin | | | | | | | |
| Marlex + 25% | 34 | 6 | 1274 | 948 | 0.18 | 0.13 | 98.24 |
| PVP | | | | | | | |
| Marlex + 50% | 23 | 3 | 1737 | 1566 | 0.11 | 0.07 | 41.00 |
| PVP | | | | | | | |
| Marlex + 25% | 33 | 5 | 1240 | 880 | 0.14 | 0.09 | 56.38 |
| Aquazol | | | | | | | |
| Marlex + 50% | 40 | 5 | 1556 | 1253 | 0.16 | 0.1 | 37.59 |
| Aquazol | | | | | | | |
| Marlex + 25% | 25 | 9 | 936 | 569 | 0.15 | 0.11 | 111.05 |
| Polyox | | | | | | | |
| Marlex + 50% | 31 | 8 | 876 | 563 | 0.15 | 0.09 | 74.32 |
| Polyox | | | | | | | |
| Marlex + 25% | 48 | 7 | 1440 | 999 | 0.16 | 0.13 | 90.55 |
| Ethocel | | | | | | | |
| Marlex + 50% | 52 | 6 | 1856 | 1625 | 0.12 | 0.09 | 61.51 |
| Ethocel | | | | | | | |

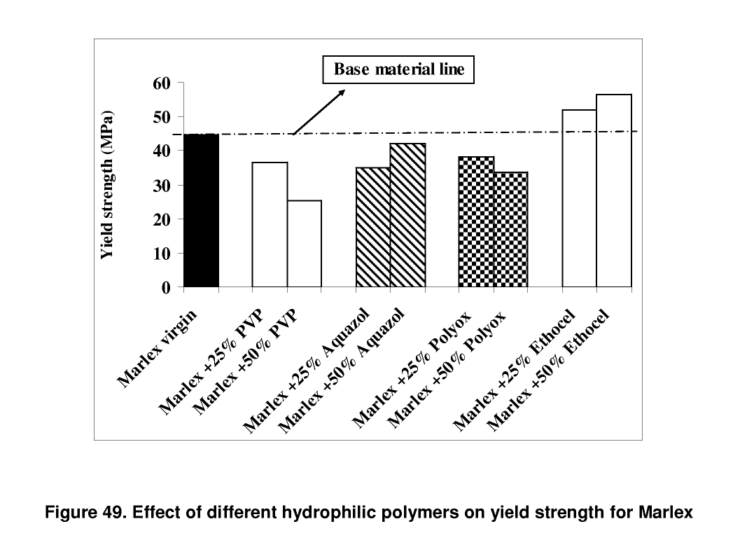

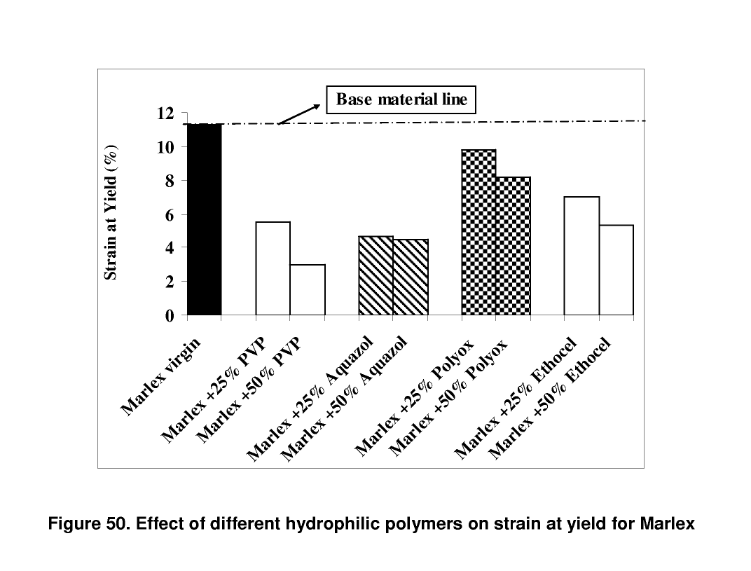

It can be seen from FIGS. 49 and 50 that Marlex in its virgin form had good yield strength and elongation at yield. With the exception of Ethocel addition of other polymers to Marlex reduced its yield strength. Elongation at yield was reduced for all materials when compared to the base resin, indicating that these blends were stiffer when compared to virgin Marlex.

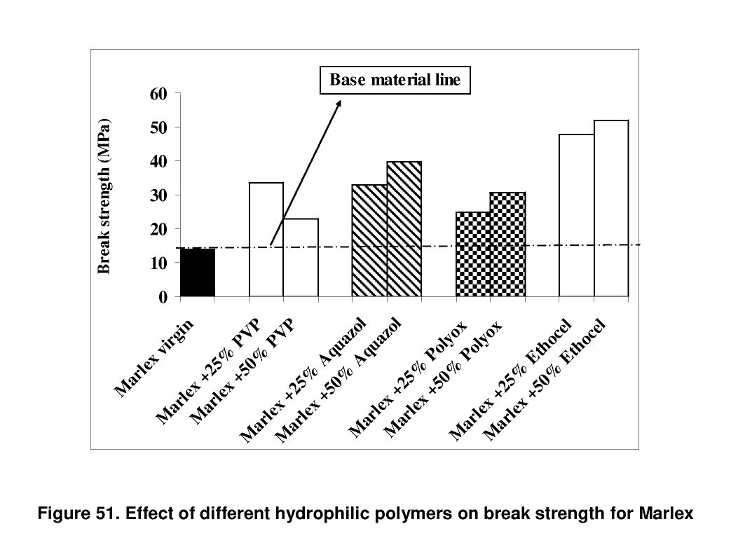

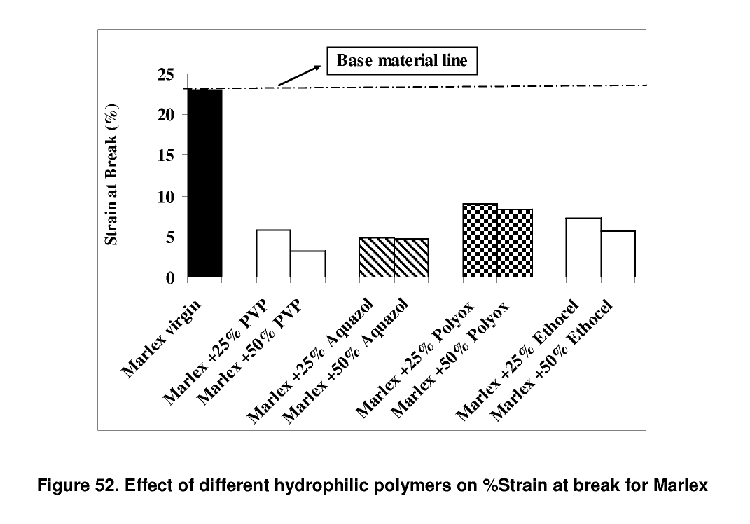

FIGS. 51 and 52 show that the break strength for Marlex increased with the addition of hydrophilic polymers, and conversely elongation at break decreased.

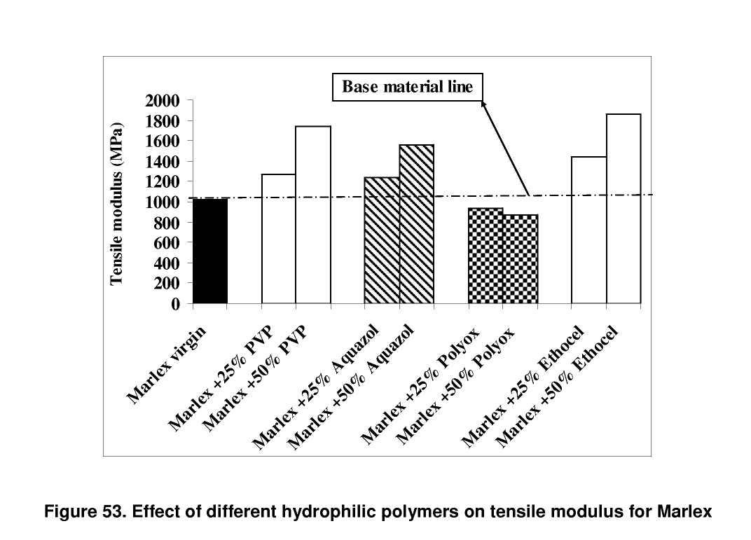

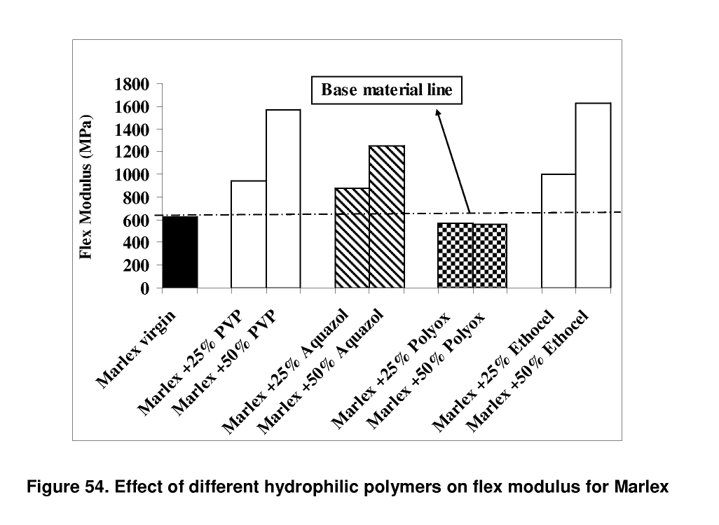

Addition of Polyox to Marlex reduced its tensile strength and flex modulus, while the other materials increased it. This can be seen in FIGS. 53 and 54 below.

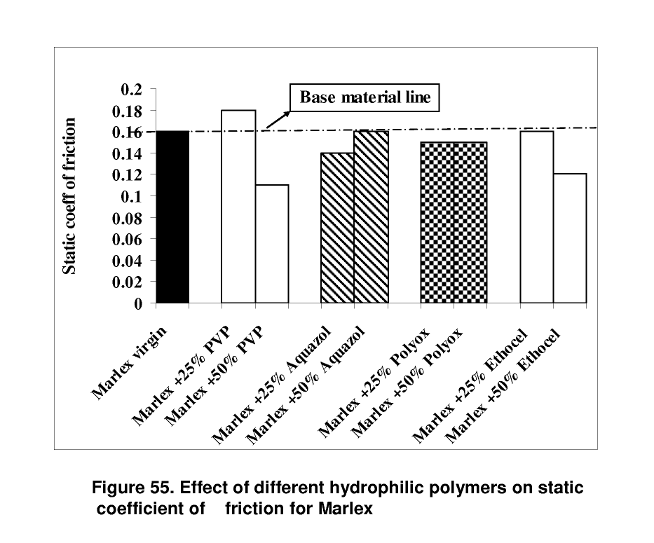

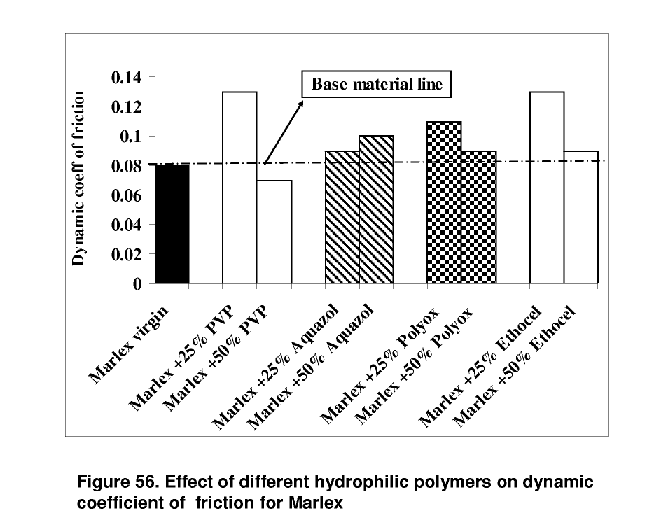

Inherently Marlex, which is polyolefin based material, has low coefficient of friction. This can be seen from the data obtained for the base material in FIGS. 55 and 56. It can be seen that static coefficient of friction was reduced when different materials were added, with the exception of PVP at 25% loading. However, dynamic coefficient of friction increased when different materials were added to Marlex, with the exception of PVP at 50% loading. This indicated that addition of different hydrophilic polymers made the surface rough which resulted in an increase in dynamic coefficient of friction. Evaluation of friction in the wet state would help in judging the use of these hydrophilic polymers with polyolefins.

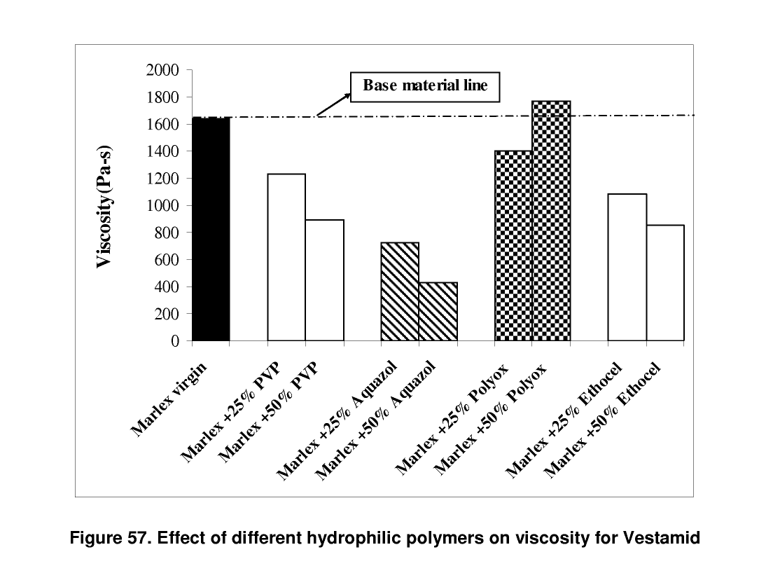

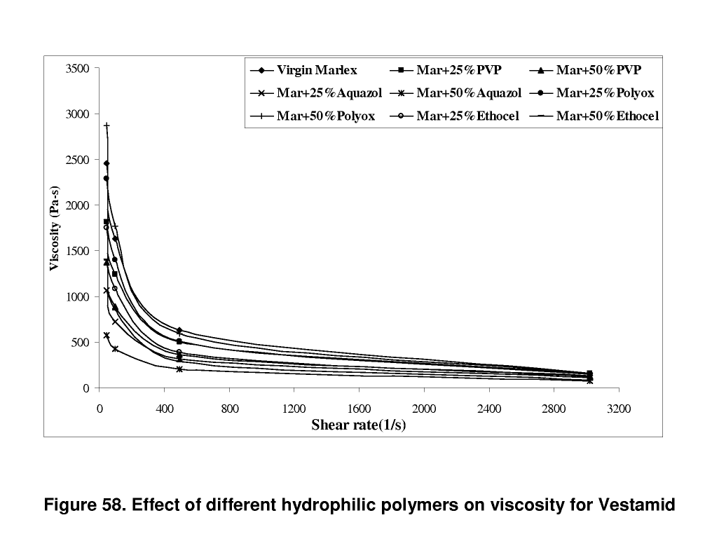

FIG. 57 shows the effect of different hydrophilic polymers on the viscosity of Marlex. It can be seen that with the exception of Polyox at 50% loading, viscosity of the polymer blends dropped down when compared to virgin Marlex. FIG. 58 shows the plot for viscosity vs. shear rate which indicated that viscosity decreased with increasing shear rate. This plots showed that these blends were shear sensitive at low shear rates but for higher shear rates the effect was negligible.

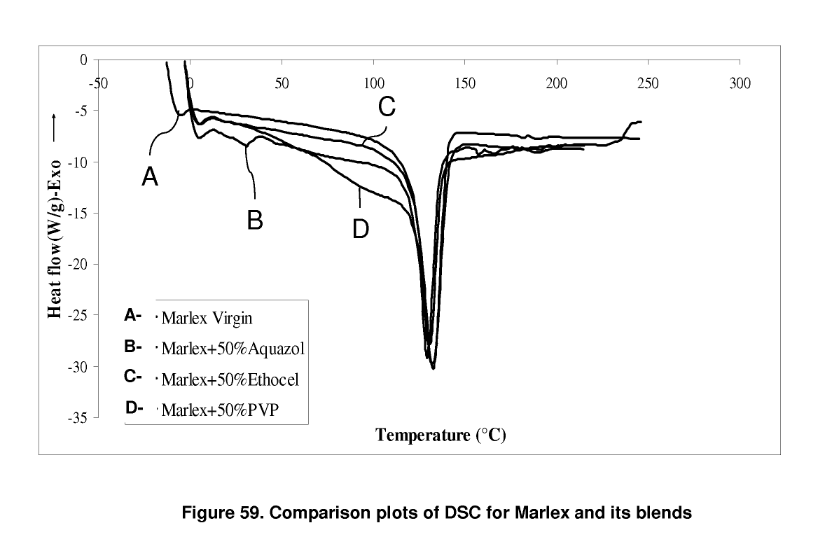

It can be seen from the DSC plot for Marlex in FIG. 59 that it had a melting point of 130° C. However, it was difficult to analyze the miscibility of different materials with Marlex since it did not show any prominent Tg values. Table 17 shows the physical state data.

TABLE 17
Effect of hydrophilic polymers on the physical state for Marlex

| | | Melting |
|---|---|---|
| Polymer | Physical state | point ° C. |
| Marlex | Crystalline | 133 |
| Marlex + 50% | 88.41% Crystallized | 129 |
| Aquazol | | |
| Marlex + 50% | 98.54% Crystallized | 132.39 |
| Ethocel | | |
| Marlex + 50% Polyox | N/A | N/A |
| Marlex + 50% PVP | 75.83% Crystallized | 129.73 |

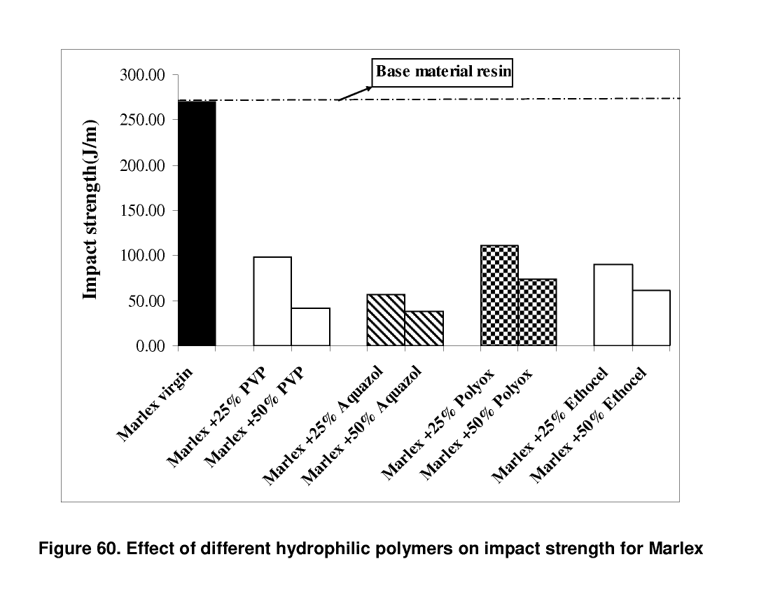

Results in FIG. 60 show that Marlex in its virgin form had good impact strength but addition of different hydrophilic polymers has reduced this value. This indicated that these blends were brittle in nature and promoted failure upon application of force. The data is shown in Table 18 below.

TABLE 18
Effect of hydrophilic polymers on Impact strength for Marlex

| Material | Impact strength (J/m) |
|---|---|
| Marlex virgin | 269.94 |
| Marlex + 25% PVP | 98.24 |
| Marlex + 50% PVP | 41.00 |
| Marlex + 25% Aquazol | 56.38 |
| Marlex + 50% Aquazol | 37.59 |
| Marlex + 25% Polyox | 111.05 |
| Marlex + 50% Polyox | 74.32 |
| Marlex + 25% Ethocel | 90.55 |
| Marlex + 50% Ethocel | 61.51 |

What is claimed is:

1. A lubricious polymer compound comprising hydrophilic polymers that are dispersed in a melt processable thermoplastic matrix.

2. The lubricious polymer compound of claim 2 wherein said melt processable thermoplastic matrix comprises a thermoplastic matrix that is capable of processing via extrusion or injection molding.

3. The lubricious polymer compound of claim 1, wherein said hydrophilic polymer are selected from the group consisting of polyethylene glycol, polyvinyl pyrrolidone, polyethyl oxazoline or ethyl cellulose.

4. The lubricious polymer compound of claim 1 wherein said thermoplastic matrix is selected from the group consisting of thermoplastic polyurethane elastomers, polyether block copolyamide polymers, polyamide 12 and polyethylene.

5. A lubricious polymer compounds comprising a hydrophilic polymer dispersed in a melt processable thermoplastic matrix having a static and dynamic coefficient of friction values of about 0.02 or greater.

6. A catheter comprising a tube including a layer of a lubricous polymer compound comprising a hydrophilic polymer dispersed in a melt processable thermoplastic matrix.

7. A wire comprising a surface layer of a lubricous polymer compound comprising a hydrophilic polymer dispersed in a melt processable thermoplastic matrix.

8. A method of applying a lubricious polymer compound comprising a hydrophilic polymer dispersed in a melt processable thermoplastic matrix comprising:
supplying said lubricious polymer compound; and
extruding said lubricious polymer compound on to a surface of a thermoplastic resin substrate.

9. The method of claim 8 wherein said step of extruding said lubricious polymer compound provides a single layer of said polymer compound.

10. A lubricious polymer compound comprising hydrophilic polymers that are dispersed in a melt processable thermoplastic matrix including a hydrophilic polymer dispersed on the surface of said compound by solution casting.

11. A lubricious polymer compound comprising a base polymer and a hydrophilic polymeric as a layer on extruded tubing.
